SEQUENCE LISTING (46 sequences)

SEQ ID NO: 1 - protein, 686 aa, Streptomyces venezuelae
MRTLLIDNYDSFTHNLFQYIGEATGQPPVVVPNDADWSRLPLEDFDAIVVSPGPGSPDRERDFGISRRAITDSGLPVLGVCLGHQGIAQLFGGTVGLAPEPMHGRVSEVRHTGEDVFRGLPSPFTAVRYHSLAATDLPDELEPLAWSDDGVVMGLRHREKPLWGVQFHPESIGSDFGREIMANFRDLALAHHRARRDAADSPYELHVRRVDVLPDAEEVRRGCLPGEGATFWLDSSSVLEGASRFSFLGDDRGPLAEYLTYRVADGVVSVRGSDGTTTRTRRPFFSYLEEQLERRRVPVAPDLPFEFNLGYVGYLGYELKAETTGDPAHRSPHPDAAFLFADRAIALDHQEGCCYLLALDRRGHDDGARAWLRETAETLTGLAVRVPAEPTPAMVFGVPEAAAGFGPLARARHDKDAYLKRIDECLKEIRNGESYEICLTNMVTAPTEATALPLYSALRAISPVPYGALLEFPELSVLSASPERFLTIGADGGVESKPIKGTRPRGGTAEEDERLRADLAGREKDRAENLMIVDLVRNDLNSVCAIGSVHVPRLFEVETYAPVHQLVSTIRGRLRPGTSTAACVRAAFPGGSMTGAPKKRTMEIIDRLEEGPRGVYSGALGWFALSGAADLSIVIRTIVLADGRAEFGVGGAIVSLSDQEEEFTETVVKARAMVTALDGSAVAGAR

SEQ ID NO: 2 - protein, 101 aa, Streptomyces venezuelae
MTEQNELQRLRAELDALDGTLLDTVRRRIDLGVRIARYKSRHGVPMMQPGRVSLVKDRAARYAADHGLDESFLVNLYDVIITEMCRVEDLVMSPSCTKEVV

SEQ ID NO: 3 - protein, 328 aa, Streptomyces venezuelae
MSGFPRSVVVGGSGAVGGMFAGLLREAGSRTLVVDLVPPPGRPDACLVGDVTAPGPELAAALRDADLVLLAVHEDVALKAVAPVTRLMRPGALLADTLSVRTGMAAELAAHAPGVQHVGLNPMFAPAAGMTGRPVAAVVTRDGPGVTALLRLVEGGGGRPVRLTAEEHDRTTAATQALTHAVILSFGLALARLGVDVRALAATAPPPHQVLLALLARVLGGSPEVYGDIQRSNPRAASARRALAEALRSFAALIGDDPDRAEDPDRADDPDRTDNPGHPGGCDGAGNLDGVFEELRRLMGPELAAGQDHCQELFRTLHRTDDEGEKDR

SEQ ID NO: 4 - protein, 199 aa, Pseudomonas fluorescens
MFKTLIIDNFDSFTYNLYQYMGQVTGEEPDVFTNDASPHDIDLGRYDCIIVSPGPGTPKRRQDVGISEDMIRDAHVPLLGVCLGHQCMAHVHGMDVDHAPEPMHGRVRHIRHNNEGVFKGLPVDMPVVRYHSLVVKALKGPFELSAWDENGMIHGIRHTERPLYGIQFHPESICTDSGLDLLRNFRDIAHRHKLERLPR

SEQ ID NO: 5 - protein, 486 aa, Pseudomonas fluorescens
MTTFDVEVRALDYNPDPLRVFRSEFLASPRHFFLESSVVKPGFSRFSFMGDSHGRLAETITYDTSSRSVRVERSDGVTREPTSDFLELMAARLNHYHCEQPQWLPFDFNLGYVGLLGYELKCETLGAQAYASHSHDAAFILATRMIAFDHAEQRCYLLYLVEHDEDRQDAAQWFDQVQARLREQPQVAEPVSRQRKLSLPQVEAWIQEHACIRHSKQRYIDKINEAQREIIDGETYEVCLTNLIEFAFADSSFDLYCVMRELTPAPHAGYFSIPDFQIISSSPERFLKIDRHHQVEAKPIKGTRPRGRCAEEDQELIEQMRGDEKDRAENLMIVDLLRNDLGQVCTIGSVRVPALFAVETYSHVHQLVSTISGQLKPSLSAVDCVRATFPGGSMTGAPKKRTMEIIDRLEEGPRGAYSGSLGWFGLGGACDLNIMIRSITVDAQVARFGVGGAITSLSDPLGEYIETMVKASGVVEAVTQLRSTSV

SEQ ID NO: 6 - protein, 420 aa, Pseudomonas fluorescens
MSLSSPHRHAVVVGILGSIGQLLANQLSIAGYSVTGIDIAVDDQSAQPHTVIQGDVLRPGNEIKQRLGDAQILVLALAQNVLSEALPQLLPSLRSDCLIVDTLSIKSEFADFVATLDVAQPMVGINPMFSGDLDPAGRPVAVVTYRDGDGDAVARLVEWLHSWPANVFQMTASEHDRTMAYLQTLGHALVMGMGLTLAESAAPLENLFELAPPPFKVMLALLARMTKNHPDVYWEIQSNNPYSQEIRSRMLAQLGKLDDRVNSGSRLDYHVSMAMLRNALKPLNPGLENTSRHLFEQLDQAPKAIEGAPESLADYRQRIDHIDDQLVDLLGQRLSLIREVAQSKKDHQTAVMQPNRVVQVVERCKARGRRHHIRESLIEQLYGLIIDEACQIEYDVIGGPRESLYEASPSAFTSSAEKTQ

SEQ ID NO: 7 - protein, 339 aa, synthetic construct
MTMITPSLHACRSMPESQLLNKITDTWYAKATVRSTPRILVPDYSSEQLIYPVARCSICEHPLVLELGPQVRSYILTQAAYQFLYGVGLLETKFVIQCCLDMLHNNIKDISDAAKLQALTVIVDEGYHAHVALDYIIQMKKKSAIEPLEVPQTNRKLDATARAYASLPESMRMDFQLLAVTLAENVLTDEVANLGRERELAQSFTTLMMDHVRDEGRHSRFFADLMKERWPQLPRATQEHFGLMLPAYLDDFLGADLSRGFERKILAHCGLTEAQAEQVIHESDPHFSTDQARMKKSILQRIYRLLNQIGVLELDSVKDAFSDRNYVTTGGGSHHHHHH

SEQ ID NO: 8 - protein, 328 aa, synthetic construct
MQIFVKTLTGKTITLEVESSDTIDNVKSKIQDKEGIPPDQQRLIFAGKQLEDGRTLSDYNIQKESTLHLVLRLRGGLFVQELASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKLEHHHHHH

SEQ ID NO: 9 - protein, 397 aa, Escherichia coli
MFQKVDAYAGDPILTLMERFKEDPRSDKVNLSIGLYYNEDGIIPQLQAVAEAEARLNAQPHGASLYLPMEGLNCYRHAIAPLLFGADHPVLKQQRVATIQTLGGSGALKVGADFLKRYFPESGVWVSDPTWENHVAIFAGAGFEVSTYPWYDEATNGVRFNDLLATLKTLPARSIVLLHPCCHNPTGADLTNDQWDAVIEILKARELIPFLDIAYQGFGAGMEEDAYAIRAIASAGLPALVSNSFSKIFSLYGERVGGLSVMCEDAEAAGRVLGQLKATVRRNYSSPPNFGAQVVAAVLNDEALKASWLAEVEEMRTRILAMRQELVKVLSTEMPERNFDYLLNQRGMFSYTGLSAAQVDRLREEFGVYLIASGRMCVAGLNTANVQRVAKAFAAVM

SEQ ID NO: 10 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 11 - protein, 309 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 12 - protein, 306 aa, synthetic construct
MDEFEMIKRNTSEIISEEELREVLKKDEKSAYIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNDIHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 13 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSSFQLDKDYTLNVYRLALKTTLKRARRSMELIAREDENPKVAEVIYPIMQVNPLNYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 14 - protein, 306 aa, synthetic construct
MDEFEMIKRNTSEIISEEELREVLKKDEKSATIGFEPSGKIHLGHYLQIKKMIDLQNAGFDIIILLADLHAYLNQKGELDEIRKIGDYNKKVFEAMGLKAKYVYGSTFQLDKDYTLNVYRLALKTTLKRARRSMELIAREDENPKVAEVIYPIMQVNPLHYAGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 15 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNPAHYQGVDVVVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 16 - protein, 306 aa, synthetic construct
MDEFEMIKRNTSEIISEEELREVLKKDEKSAAIGFEPSGKIHLGHYLQIKKMIDLQNAGFDIIIVLADLHAYLNQKGELDEIRKIGDYNKKVFEAMGLKAKYVYGSEDHLDKDYTLNVYRLALKTTLKRARRSMELIAREDENPKVAEVIYPIMQVNGIHYSGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 17 - protein, 306 aa, synthetic construct
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEWMLDKDYTLNVYRLALKTTLKRARRSMELIAREDENPKVAEVIYPIMQVNGAHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 18 - protein, 315 aa, unidentified
MNASDYRSIAEDWEHRATIRTRPRRVLENDDNLIYPLCRQPLVACATFLEHCSQWRDFVLVQSFYKFINDVVIFETEIVDKTARSIAKNRFAVPFPLACRYDAMTIVVDEDYHALVALDFLQQTVEMTGIQPLELPKQIELGRALPAAQAQAPAHLHDAVELIAVAIAENTVTHDVAAFAKDDSVKSSVRGMMADHLSDEGRHAQFWTRLVRLYWQVASPQDRDSIALVLPTFLAQYLTNDLQASFDLQLIEHLDVSPGIRHALREEINALDFPITRQHPLIGNITRFLHHSGLLQTPSVAQALDDYLPPAGSAA

SEQ ID NO: 19 - protein, 309 aa, unidentified
MNAVDYCSFADAWEERATIRTRPRRQVEDDQRLIYPLSRQPLVLSATFVRECPQQRDFALVQSLYKFIHDVVIFETEIVNHTASAIARGRFAHDFPFACRYDAMTVVVDEDYHALVAMDFMQQTIELTGITPVALPSEIELSRAIPAALAQVPERLRQAAELICVAIAENTLTNDVAAFARDTSVKESVKGLMADHLQDEGRHATFWQRLVRIYWQAAPADDQASLAHMLPTFIERYLTNDIQKAFDLALIEHLHPDPAVRQALREEAQGLAFPISRQHPLLGNILRFFKGSSMLDSPCVRDALVAYLP

SEQ ID NO: 20 - protein, 315 aa, unidentified
MKAEDYVSFVDAWEARATIRTRPRRIVEDDERLIYPLSRQPLVLSETFTRQCPALRDYALVQSLYKFINDVVIFETEIVDKTARSIAKDRFAIRFPFACRFDAMSVVVDEDYHALVAMDFMQQTIALTGIEPITLPTQIELSRAIPAALAQAPEHLRSAVELICVAIAENTVTHDVAAFAKDDTVKQSVKGLMADHLLDEGRHSGFWTRLVRIYWQTAAPADRAAIAQILPVFIDQYLTNEIQNGFDFILINHLPVAEVVKQALKAETLALSFPISSHHPLVGNIVRFFRNSTMLEAPCVRQALVDYLPAGEVYA

SEQ ID NO: 21 - protein, 334 aa, unidentified
MTDHQAANRRAARLRPPELPEHDAADPAAAAVIRGLTRSWPRRAAVKKTEPDRDALFEPARDDFPDTLLPFHTHPRYAALDEPARARLRAWGWIAYNKNVIDIEQYVVNPGFGLITSDVFGLAPPDTVVLGVMQAMVDEQYHTLMHQNANALTRRRRGLTLPDAVLPACRTLRAHRAAVQRATDPRHEALVQLAFTTVAETSISAYLALFIGDETVQPVNRATVSVHRRDELCHATLTGELFTTVFTELDAADRERLFEALAAGIDAFTGGDMTTWAAIVAHEGIPGGREMIADTADATAHQPVVQDCSAIHRLCASVGAGEDFDAHRARRLQT

SEQ ID NO: 22 - protein, 290 aa, unidentified
MLEKLSVLWSTRAAVNNKQPTYSELAFDPGKNDFCESLLPFRNHPAWIEASPEMKSTCLSYAWIIYNLKTIYIECDIVTPACEDIIKTPPSATRNRALLQESISEALLDEALHTKMSVTACNYIYHHRDLAHLNFTDFNLIKWKNWVLSSCSAEWERRLTRFGIACASETLITDYLKTLAEDSSVQSICHQVTKTHAMDEWSHSSVFSYVAADLISTLSQHEKTHLKAIIKKTVSMFANNEMGAWEMAFSHARLPRYADIIHDTATLGEVNVYTESVDKLLSRIGLEETH

SEQ ID NO: 23 - protein, 290 aa, unidentified
MDRLNCGTDDVVKNMLAKLSGLWKTRAAVNNPQPDYWGLAFDPNKQDFSLSLLPFRHHQAWLEAPQDLQSKCLSYAWGIYNLKTIYIECDVVVPACEDIIKTPPPSQNRALLQDVMSQALLDEALHTRMSIMACNYIYDMRKLTPLDFSAFNLVTWREALLANCTAEWERRLTRFGVACASETLITDYLKTMAEDTSIQAVCHEVTRTHAIDEWSHSSVFSFVASDIVHGLSRTEREYLRAIILRTVQMFANNEMGAWARVFSMLQMPHASEILHEVGDNNEAPNTPSKP

SEQ ID NO: 24 - protein, 319 aa, unidentified
MTITSDAPGPAGSGPDTLRRLVAAWPRRATIRTDLDKVTDPGPYDPALMDYPVSLLPFAGHPSYLAASDEQRLQVNTLAWLAYNERVIAAEEHVANPTFEKLSHGVFPGADGVDAREAIQQSHIDEVWHTYMHMLAMQRSREARRVTAEPNDYLHPVTNRRLYRLADEASEQWERDLLYLLWTVVGEVSINAFLDLVAGDRTIQPGHALIARLHARDEAAHGPILAEVMKDVFVHLNAAQREVLVRRLPEAVTAFGAEDYELWLRILDFAGFADARAIVEDSRALPGAELMLTDFAPIQRLIRELGVDDRVDLDLPAPS

SEQ ID NO: 25 - protein, 341 aa, unidentified
MRYEESGVPGGLTTWAARGWAEEHGIGSATLRRLVRAWPRRAAVTTRPEVMEESAAYDPALPDYPLEIVPFAEHPRFLAARPDQRRQVLTGLWIGYNERVIATEHLIAEPAFDLVMCGVFPGGDDPLIRQSVQQAVVDESFHTYMHMLAIARTRELRDVRTRPEQPPLVTYRRLCALLATMPEEWERHVAVLVWGAVAETCINSLLALLARDESIQPAHSLITTLHLRDETAHGSVVTEVVRELYARMNAAQRRTLVRCLPLALEAFAEQDLSTLRLELATAGVHGADEIVGDLRATSTGRRLVRDFSGAQRMVEQLGLAGEVDFDFPERPAWSPAAATSH

SEQ ID NO: 26 - protein, 318 aa, unidentified
MSALSREPVFQFSLNDWNTRASVRTSTHDYRLPANVQEQLETRHWFPPTFLPYLAHPVIEAAGRSMLHRLTARHLVHFLDYTTLLEHRIVNRAVETIVHGELPVAVPAPMKTAALQLYTDEGYHALFSNQVAEQIASLYGITKRPVMPRRITRMNLLIARTDQQQRPLACFLLGFVSETIIARELLDVCRDSLVSGVNDMLRDHLTDEARHSRYFTEVFHYLWLHLDPRQRTFTATTLLDILEIFFEVDEHWLQESLCGADIAGTTVMEILGTITTAQASRQRARTGSLATMSALKKAGFFAEPQNQTLFARAGLIDG

SEQ ID NO: 27 - protein, 320 aa, unidentified
MISTNSQAAAAEHDNVSRWYANATVRHRPRIMVPDSSKGEFIFPRSRQLLSEHPLVTARGHDAVAYVLAQSAYKYMYEIGLLETRFVIDCSLNIVNNQIKGFNDDEKREALTIVIDEGYHAYVALDFIIQMKSLTGIQPIEVPETNGNLQAVSQAMDALPAFMHNDFQLISVCLAEHTLTKDLLSIGREKDATQAFTQVMTDHVSDEGRHANYFARLMKQHWVSLPDATKTTIGFMLPNYLDEYLAGDGERSFDKKVLLALGMSEAEAMRVIDDTHPAFMDNLHSYVSVTKANLVKLLARCGVLDHAPTRASFVAHGVEL

SEQ ID NO: 28 - protein, 319 aa, unidentified
MQTAIEIAKVDHQLIVRLNQAWTRRATVCSPDKWSVDEVFDPARPDYPEGMVPFFHHPKFQALSEALKSRVVTWGWIGYNLRTVTAEEHVVNPALSVIANQYLGKDDWHFREAMQQTLIDEHYHTLMHLRAIERTRLERALDQDLDLPPSVTYLRLLALRETLTEQWQKDLAAITFAVVAEISVNAYLDLLADDQTIQPQNRRVAEQHNRDEYAHSKVLAEVAKVMYGNMQPPQRAFFARTLSQALSAFIAQDYSMWEAILTQLGVEDAAQIIADTRESNKNATIMRDYSGLHRLAQDLGISEQIDFDFRPTLKTTAAA

SEQ ID NO: 29 - protein, 308 aa, unidentified
MMTLQSYKSHAESWEQRATIRSRPRRIVEDDGLHYYPLERQPLCAHPAIVDAGDAVRDFILLQSFYKYINDVIIFETEIVNATALKIARGRFAHDFPFACRHDAMSVVIDEDYHAYVAMDYLRQVEVHTGVAALAANHEIELSRAIPRALERVAPPYRDGMELLAVAISENTVTAEVAAFSRDTTLKRSVKGVMSDHLADEGRHSVFWINLVKLYWSEIDEAARSALGETLPVFLKEYLSAELQLAFDRRLIDALDLPAATRREIAGDMVGAYPITSQHPMIVNIRNFFKLSGLLDHAPTRAALASVL

SEQ ID NO: 30 - protein, 316 aa, unidentified
MSPRDYKSKELVWDSLAATRSKPRRVLPEGDVVGHLYPPAKAALLTHPLMKNLPPETLRLFFIHSAYKFMGDIAIFETETVNEVAMKIANGHTPITFPDDIRHDALTVIIDEAYHAYVARDFMRQIEQRTGVKPLPLGTETDLSRAMAFGKHRLPETLHGLWEIIAVCIGENTLTKDLLNLTGEKSFNEVLHQVMEDHVRDEGRHAVLFMNVLKLVWSEMEESARLAIGQLLPEFIREYLSPKMMAEYERVVLEQLGLAAEHIERILSETYSEPTLEDFRARYPLSGYLVYVLMQCDVLSHAPTREAFRRFKLLAH

SEQ ID NO: 31 - protein, 332 aa, unidentified
MARMMNNAKSKIGEQILTLSKDGLTTGFTRQLQIEWEKRATVRSRPKVVYDKEAKGYYYPISRTPLAIHPTVMAKGERAIEFLLIQSLYKYSNDIATIETRVVNETIIKVLTNQMPIEFNKEQKINLYTIMVDEAYHAYVAFDAMLQIEDHTGVEALPLPEVIEIEKAITKVKEKLDNKYHNIFDLICVCLAENTLTKEIITMTDQEETHPFFQKIIKDHFADEKRHSGIFYKLLEFIWINLNEEYKEHIGEIIVDFIEEYLGLTIQINFDKQVLSKIGFSDKEASKIVEDTYGGFKLTKDHPMLKNIIMLLDKAGVFDQFTLPKFEEKEWA

SEQ ID NO: 32 - protein, 319 aa, unidentified
MFQHSTQDDLYRSNDATDERLIKIERSWHKRAQIKLDSQRPDIQLDLQAPDFLEALLPFAHDPHYQNYPHALKLLILSAGWVIYNQKTIAIETEIICPSCIDLLQLGSRLSSASAATISETLADEAYHTLFSINMCELAITQRGMRIKWPELELTRHLSQAQATLSEADFKIYRLAFSLVSETFISDYLADLSDAPEIQPVFRHVVALHKKDELVHKHIFPLFVQQVVGGFNAQQTALFTRGIANSIRIFPMREISAWRIILPQLLEVFGDQSLLLPTQFTETGEADYSAMPEILSSIGIHPEAVLPEPQMMSEPAITV

SEQ ID NO: 33 - protein, 312 aa, unidentified
MSLDQINDHWYAKATVRSTPRIIVPDYDGEQLIYPVSRCAMCEHPIIQAHGKRIKNYVLTQAAYQFLYNVGLLETKFIIQCCLDMLHDKFPGIGEVEKLQALTVIIDEGYHAHVALDYLTQMNEKSGIEAIEVPQTNQKLDATARAYAQLPESLRTEFQLLAVTIAENVLTDEVANLGRERELSQSFTTLMVDHVRDEGRHSRFFADVMKKRWPTLPLSTRERFGMMLPAYLDDFLNVDPERRFERRMLAHCGLSPEQAEQVIHESDPTFHADHARMKKSILQRLYRLLRQMGILDLPQVREAFADRDYVSA

SEQ ID NO: 34 - protein, 315 aa, unidentified
MTGHLPEHDASDPVESAVIDGLVRSWPRRATVRRTEPELDIPTMDDLYDDTREDYPEVLLPFAGHPSWERLDEDVRTRVRAWAWIAYNKNVVDIEQDVVTPGFGLLFRDVFGTGFSDADRAAVVQSMVDEEYHTLMHLNASALTRRRRGWALPDTTLPESSTVRRHRQARAQAESPRAAALTTLAFATVAETSIADYLSLIAGDQTIQPVHQATIALHRRDERAHASVATEMIAMVHDKLDSDDRSFLARALRDGVDAFTATDGAVWSAILAAEQVPDADQMLRDVAEDPDRRHFLQDCSAINRLLARFGELEHR

SEQ ID NO: 35 - protein, 311 aa, unidentified
MSLIETTLKKWDRAAIVRSRPRRLLNEQDSQSTLFFPLERLPLCLHPRVAALGEQALRYIQVQTLYHYLNGIANIELDIINDSSYTLYKNAVGVDFPLEMRLDALTVVVDESYHALVALDLINQVEQMTSIAPLPMPKDTEASYALAQSLAMGDERLRELLRLVCVSLSEQALTTDLIDVINNEDIFPTFYLVMKDHVADEGRHARFSQQVLSHIWRHSDAATKQALQQPILAFLEGYATDAIAKDFSACMLRQLGLTEAEIIDIIADTYPATHVKPLETNNIVTEQMLVALNKSGILADLPIADALHATI

SEQ ID NO: 36 - protein, 329 aa, unidentified
MTTMSAPAIATGGTDAAHQHEQSEVKALLQRIGDGWIKRAKVRRDAVDLAFDPARPDFLEELLPFHAHPLYQRLAPELKSKILSAGWIIYNEKTVAIESAIVSPSCYDALDGRIPGLTDETSRQIVCETLVDEAYHLLLVANANRLTRARRGLEDLKIPSFNLVTMMNREKSLQSEDWQKILVHLATSVVSEIFVSDYLHQLSDCAEIQPMNMATVAAHRHDELAHSKIFTLMTKTFYPALNPREQAFFASVLPKPIEWFADLELDIWSSVLEQLRVPGAATIIADCRPLNAAARERLDYTGIVGLSHEVGILSTDAGRNSFAAQGIAI

SEQ ID NO: 37 - protein, 309 aa, unidentified
MTAAREVPEYERDLLVQLTLRWGKRVAVKKDELDLDGHFDAALPDFPEHLVPVLGLPGADRLDRDARERILSAAWIAYNAKTAAIENEIILPACRLMLQERIPVRRDDVAVDALHQTIIDEHYHILMCHNAVGVTRRRRGLEDVRFAPGVWSVVQGLNAVRAGLSGFDRDIVDIAFSLAAETTISGFLSALATAQEIQPMNRITTDLHRRDESGHAVVFRELCGSLYRNLDGAQQRTFREALVTGLTAFRSADLEPWVTVAAQGGFEIDTGQLSEWAASRPVPARDTGPLQLLLDDLGISHDLVEMVQR

SEQ ID NO: 38 - protein, 327 aa, unidentified
MTLTASIPGTQKTSQSRKLLSKMASNWSRRAKVVQAKKQNFENNSELVFDSARDEFRIDLLPFKDHPAFLEAPPEMQKQILSCGWIAYNEKTVDIESKVISPACSHIIYQEVPGSDDGIGREIASDTLVDEAYHILLVMQACRITREKRGLESLKLPSFNLVKQMTLEKAKYSERWQKILVQIVTATVSEVFISDYLNLLAKDTNIQPMNRLIVDTHRQDEMAHHSIFINLAKCIYSELSPQQQEFFADILPKAVRWFANSELEVWQTMLEYLGFSKTREVIADCAVTDEVNLSRIDYSAVVLLAKELGVLDSQKGIDSFARQGLLK

SEQ ID NO: 39 - protein, 312 aa, unidentified
MSLDLINDHWYAKATVRSTPRIIVPEYDGEQLIYPVSRCAMCEHPIIQTHGKHIRNYLLTQAAYQFLYNVGLLETKFIIQCCLDMLHDKFPGIGNEEKLQALTVIIDEGYHAHVALDYLTQMNEKSGIEAIEVPQSNQKLDATARAYAQLPESLRTEFQLLAVTIAENVLTDEVANLGRERQLSQSFTTLMVDHVRDEGRHSRFFADLMKKRWPTLPESTRERFGLMLPAYLDDFLDIDPERRFERRMLAHCGLTPEQAEQVIHESDPTFHADHARMKKSILQRLYRLLRQMGILDLPQVREAFADRDYVSA

SEQ ID NO: 40 - protein, 314 aa, unidentified
MLIAEQNEITAQWYQRATVRYTPRIIVPDYSDEELIYPLARCAVCEHPLVLERGQATRAFILAQAAYQFLYGVGLLETKFVIQCSLDLLHNQIPGIGEFDKLQALTVIIDEGYHAHVALDYIMQMQHQSGITPLQVPESNHKLDAAARASATLPPAMRPDFQLLAVTLAENVLTEEIATLGRDKGLAQTFTTLMMDHTRDEGRHSGYFADLMKKRWTQLPQATRDQFTQMMPDYLEDFLGTDTTRQFERRILRACKFTPQQIEQIIAESESEFIDLHRQLTQKTRIRLDRLLKQIGVREPDTNTTATAHASDAA

SEQ ID NO: 41 - protein, 320 aa, unidentified
MNQAQENHPAPDGAISAWYEKATVRYTPRIIVPDYTTDALIYPTALSIISEHPLVVARGKEIRSFILTQSAYRFLYGVGLLETKFVIQCSLDLVHDRIFPVDDMQRLQALTVIIDEGYHAHVALDYIVQMREKSGIVPLSVPQTNRKLDATARVYATLPPDLRMDFQLLAVTLAENVLTNEIATMGREKELTKSYITLMTDHVMDEGRHSNYFNDLMRKRWAQLDATVQSTFGTLLPAFLDDFLGIDLHRAFEREMLDACGFQSSDVQRILEDTQETYSNNHATLSTRTKARLFRLAKTFGILDRDDVRDAFAARGYAFQ

SEQ ID NO: 42 - protein, 313 aa, unidentified
MLIAEQNDLTNHWYQRATVRHTPRIIVPDYSDEELIYPLARCAVCDHPMILARGQATRAFILAQAAYQFLYGVGLLETKFVIQCSLALLHHEIPGIDEFARLQALTVVIDEGYHAHVALDYIIQMQARSGIVPLQVPQSNHKLDAAARASATLPEAMRADFQLLAVTLAENVLTDEIANLGRDKGLAQTFTTLMMDHVRDEGRHSNYFADLMKARWAHMPQATRDRFTAMLPDYLDDFLGTDPSRSFERKILRACEFSPAETERIIDETQGEFQALHDQLTAKTKIRLDRLLRQIGVPDPANADSAAALDAAV

SEQ ID NO: 43 - protein, 324 aa, unidentified
MGCDAPGQEKPMLIAEQNDLTNHWYQRATVRHTPRIIVPDYSDEELIYPLARCAVCDHPMILARGQATRAFILAQAAYQFLYGVGLLETKFVIQCSLALLHHEIPGIDEFARLQALTVVIDEGYHAHVALDYIIQMQARSGIVPLQVPQSNHKLDAAARASATLPEAMRADFQLLAVTLAENVLTDEIANLGRDKGLAQTFTTLMMDHVRDEGRHSNYFADLMKARWAHMPQATRDRFTAMLPDYLDDFLGTDPSRSFERKILRACEFSPAETERIIDETQGEFQALHDQLTAKTKIRLDRLLRQIGVPDPANADSAAALDAAV

SEQ ID NO: 44 - protein, 320 aa, unidentified
MNQAEENHPAPDGAISAWYEKATVRYTPRIIVPDYTTDALIYPTALSIICEHPLVVARGKEIRSFILTQSAYRFLYGVGLLETKFVIQCSLDLVHDRIFPVDDVERLQALTVIIDEGYHAHVALDYIVQMRAKSGIAPLSVPQTNRKLDATARVYATLPPELRMDFQLLAVTLAENVLTNEIATMGREKELTKSYITLMTDHVMDEGRHSNYFNDLMRKRWAQLDATVQSTFGTLLPAFLDDFLGIDPHRAFEREMLGACGFPSGDVQRILDDTQETYSGNHAAVSTRTKARLFRLAKTFGILNRDDVRDAFAARGYAFQ

SEQ ID NO: 45 - protein, 312 aa, unidentified
MSLDLINDHWYAKATVRSTPRIIVPDYDGQQIIYPVSRCAMCEHPLIREHGPELKAYILTQAAYQFLYNVGLLETKFIIQCCLDMLHDKFPGIGDTEKLQALTVIIDEGYHAHVALDYLTQMNAKSGIEAIEVPQTNQKLDATARAYAQLPESLHKEFQLLAVTIAENVLTDEVANLGRERELSKSFTTLMTDHVRDEGRHSRFFADLMKKRWPTLPESTRERFGLMLPAYLDDFLDIDRERRFERRILAHCGLSPEQAEQVIHESDPTFHADHARMKKSILQRLYRLLRQMGILDLPQVREAFANRDYACA

SEQ ID NO: 46 - protein, 322 aa, unidentified
MCPPHLTGKKMSLDLINDHWYAKATVRSTPRIIVPEYDGEQLIYPVSRCAMCEHPIIQAHGKHIRNYLLTQAAYQFLYNVGLLETKFIIQCCLDMLHDKFPGIGNTEKLQALTVIIDEGYHAHVALDYLTQMNEKSGIEAIEVPQSNQKLDATARAYAQLPESLRTEFQLLAVTIAENVLTDEVANLGRERELSQSFTTLMVDHVRDEGRHSRFFADLMKKRWPTLPESTRERFGLMLPAYLDDFLDIDPERRFERSMLAHCGLTPAQAEQVIHESDPTFHADHARMKKSILQRLYRLLRQMGILDLPQVREAFADRDYVSA

(19) United States
(12) Patent Application Publication
(10) Pub. No.: US 2026/0028653 A1
(43) Pub. Date: Jan. 29, 2026

(54) BIOSYNTHESIS OF PARA-NITRO-L-PHENYLALANINE

(71) Applicant: Nitro Biosciences, Inc., Newark, DE (US)

(72) Inventors: Aditya Kunjapur, Newark, DE (US); Neil Butler, Newark, DE (US)

(73) Assignee: Nitro Biosciences, Inc., Newark, DE (US)

(21) Appl. No.: 19/342,324

(22) Filed: Sep. 26, 2025

Related U.S. Application Data
(63) Continuation-in-part of application No. 17/774,302.
(60) Provisional application No. 62/930,720, filed on Nov. 5, 2019.

(51) Int. Cl. C12P 13/22; C12N 9/00; C12N 9/02; C12N 15/70; C12P 21/00

(52) U.S. Cl. CPC C12P 13/222; C12N 9/0083; C12N 9/93; C12N 15/70; C12P 21/00; C12Y 601/0102; C12N 2800/101

(57) ABSTRACT

The present invention provides a recombinant cell for producing para-nitro-L-phenylalanine (pN-Phe). The recombinant cell comprises heterologous genes encoding heterologous enzymes. The recombinant cell expresses the heterologous enzymes and contains a native metabolite. The native metabolite is converted to the pN-Phe in the recombinant cell. The biosynthesized pN-Phe may be incorporated into a target polypeptide in the recombinant cell without requiring exposure of the recombinant cell to exogenous pN-Phe. A cell culture comprising the recombinant cell is also provided. Further provided is a method of producing pN-Phe by a recombinant cell comprising heterologous genes encoding heterologous enzymes. The method comprises expressing a native metabolite by the recombinant cell, expressing the heterologous enzymes, and converting the native metabolite to the pN-Phe in the recombinant cell. The method may further comprise incorporating the pN-Phe into the target polypeptide in the recombinant cell.

### CROSS-REFERENCE TO RELATED APPLICATION

This application is a continuation-in-part of U.S. application Ser. No. 17/774,302, filed May 4, 2022, which a U.S. national phase application of International Application No. PCT/US2020/059094, filed Nov. 5, 2020, which claims priority to U.S. Provisional Application No. 62/930,720, filed Nov. 5, 2019, and the contents of each of which are incorporated herein by reference in their entireties for all purposes.

### REFERENCE TO SEQUENCE LISTING

The present application is being filed along with a Sequence Listing in electronic format. The Sequence Listing is provided as a file entitled 2103933-000008_SubstituteSequenceListing.xml, created Oct. 9, 2025, which is 54,000 bites in size. The information in XML file format of the Sequence Listing is incorporated herein by reference in its entirety.

### FIELD OF THE INVENTION

The present invention relates in general to biosynthesis of non-native non-standard amino acid para-nitro-L-phenylalanine (pN-Phe) in a recombinant cell and uses thereof.

### BACKGROUND OF THE INVENTION

Live bacterial vaccines in the form of attenuated pathogens or recombinant delivery vehicles are promising technologies for prevention of widespread diseases. However, these in situ antigen-producing platforms are often limited by their inability to elicit long-lasting immune response when attenuated or when provided at low doses required for safety. Coupling these technologies with the biosynthesis of a precise immunostimulant could overcome a major hurdle to vaccine development for several types of pathogens by triggering high, sustained humoral response with low bacterial administration. The molecule para-nitro-L-phenylalanine (pN-Phe) has been demonstrated to act as an immunostimulatory compound when present as a surface residue on multiple proteins, including self-proteins for the purpose of breaking immune self-tolerance. It has been demonstrated that pN-Phe incorporation within a protein antigen and subsequent immunization using this modified antigen leads to formation of antibodies that predominantly bind to other regions of the protein antigen rather than the pN-Phe containing epitope, thus cross-reacting with wild-type antigen. Although pN-Phe can be incorporated site-specifically within proteins in live cells, there is currently no means to biosynthesize pN-Phe within live cells. Thus, pN-Phe incorporation cannot yet be used to enhance live vaccines.

pN-Phe has compelling use cases given its immunochemical properties. In developing immunotherapies against cancer and autoimmune disease, target antigens are commonly self-proteins that are upregulated in diseased cells. Generating a strong and sustained immune response toward these antigens is difficult, given that CD4⁺ T helper cells, which are required for MHC Class II response, are tolerant of self-proteins. This tolerance inhibits the activation of B cells for autoantibody production. However, the introduction of pN-Phe into self-proteins has demonstrated termination of T cell tolerance in both mouse models and human cell lines via the generation of immunogenic pN-Phe epitopes. When this strategy was applied to the cytokine TNF-α, a high and sustained IgG polyclonal antibody response was measured lasting over 40 weeks. Notably, the response targeted multiple regions of TNF-α distinct from the pN-Phe epitope. Testing indicated protection against endotoxemia in mice following immunization with pN-Phe-TNF-α, demonstrating that pN-Phe modified epitopes can generate physiologically relevant and wild-type targeting antibody response. This strategy has since been applied to other antigens relevant to autoimmune disease, such as C5a, and tumors, such as PDL1 and HER2, resulting in T-cell mediated activation for autoantibody response.

The possible immunochemical applications of pN-Phe would be expanded by coupling its biosynthesis with live bacterial vaccine technology. While progress has been made in developing bacterial vectors for antigen delivery, a persistent challenge in bacterial vaccine engineering is a lack of methods to tune immunostimulatory mechanisms. Engineering bacterial strains to express pN-Phe in antigen epitopes could potentially both modulate immune response and promote sustained antibody production, given precedent with purified pN-Phe containing antigens. If bacterial vectors could introduce pN-Phe into antigens in situ, it could aid in enhancing antitumor or antipathogen CD4⁺ T cell response by mitigating the issue of tolerance.

To address the threat of pathogenic disease in the era of increasing antibiotic resistance, vaccines that can stimulate the appropriate form of immune response toward desired antigens must be developed. Engineered bacterial vaccine vectors offer a platform for immunization against both native and heterologous antigens with immunological benefits. Attenuated pathogenic bacteria can directly target mucosal antigen presenting cells (APCs) due to virulence factors which elicit tropism. For example, Listeria monocytogenes can be directly internalized by APCs, wherein it can translate antigens for MHC class I and II presentation, enabling CD4⁺ and CD8⁺ T cell response. E. coli can also be engineered to selectively invade nonphagocytic cells, eliciting systemic protection against the model antigen ovalbumin after oral administration. Bacteria that express heterologous antigens associated with cancer have reached phase III clinical trials, with recent failings due to limited efficacy rather than safety. Non-pathogenic, commensal bacteria such as Lactobacilli have also been developed as vaccine vectors due to high safety and demonstrated mucosal delivery and immunostimulatory behavior. However, native mucosal tolerance of these bacteria may limit heterologous antigen immunogenicity. Overall, in the toolset to balance tradeoffs between safety and immunogenicity, there are tools to attenuate vectors (virulence factor knockouts, engineered auxotrophy, etc.) but few options to enhance long-lasting immune response. For some pathogen immunization platforms, immunomodulating tools such as production of modified antigens that contain pN-Phe could provide the immunogenic enhancement needed for broad, sustained immunity.

As a non-standard amino acid (NSAA), pN-Phe has other potential uses that have been demonstrated. Naturally-occurring (standard) amino acids (SAAS) are the 20 unique building blocks composing all proteins derived from biological systems. Non-standard amino acids (NSAAs) have been developed bearing functional groups beyond those encoded by the 20 standard amino acids. To date, more than 70 non-standard amino acids (NSAAs) have been developed for in vivo protein translation. Non-standard amino acids (NSAAs) can be added to protein sequences using multiple approaches, including site-specific incorporation and residue-specific incorporation. Non-standard amino acids (NSAAs) have also been introduced within polypeptide sequences in vitro using flexizyme technology and other approaches. Non-standard amino acids (NSAAs) can also be added to peptide sequences using chemical strategies, such as solid-phase peptide synthesis. Prior to the development of in vivo NSAA incorporation techniques, pN-Phe was incorporated into peptides or proteins using solid-phase peptide synthesis for its properties as a chromogenic peptide substrate and an electron acceptor. When in proximity to excitable fluorescent structures such as pyrenyl, tryptophanyl, or anthraniloyl groups, nitrophenyl groups facilitate energy transfer, thereby preventing photon emission. Thus, nitrophenyl groups can be incorporated into proteins or peptides to serve as distance markers between pN-Phe and an excitable group to characterize protein landscapes. While applications of this technology have been limited to tryptophan distance probes and electron transfer mapping, there is high potential for pN-Phe probes to simplify binding assays more broadly. In addition to fluorescence quenching, pN-Phe has served as an internal protein IR probe and an enzymatic activity enhancer.

pN-Phe is not known to occur in nature and is demonstrably foreign to well-characterized bacterial models such as Escherichia coli and yeast models such as Saccharomyces cerevisiae.

There remains a need for recombinant cells producing para-nitro-L-phenylalanine (pN-Phe) and/or target polypeptides having the pN-Phe.

### SUMMARY OF THE INVENTION

The present invention relates to novel recombinant cells producing para-nitro-L-phenylalanine (pN-Phe) from a native metabolite. The inventors have engineered a metabolic pathway enabling cells to produce pN-Phe and introduce the pN-Phe into target polypeptides in the recombinant cell. A recombinant cell for producing para-nitro-L-phenylalanine (pN-Phe) is provided. The recombinant cell comprises one or more heterologous genes encoding one or more heterologous enzymes. The recombinant cell expresses the one or more heterologous enzymes and a native metabolite. The native metabolite is selected from the group consisting of chorismate, para-amino-phenylpyruvate (pA-Pyr) and para-nitro-phenylpyruvate (pN-Pyr). As a result, the native metabolite is converted to the pN-Phe in the recombinant cell.

In the recombinant cell, the native metabolite may be the chorismate, the one or more heterologous enzymes may comprise PapA, PapB and PapC, and the chorismate may be converted to para-amino-phenylpyruvate (pA-Pyr) in the recombinant cell.

Where the native metabolite is the chorismate, the recombinant cell may further express an N-monooxygenase, and the pA-Pyr may be converted to para-nitro-phenylpyruvate (pN-Pyr) in the recombinant cell. The recombinant cell may further express an aminotransferase, and the pN-Pyr may be converted to the pN-Phe.

Where the native metabolite is the chorismate, the recombinant cell may further express an aminotransferase and the pA-Pyr may be converted to para-amino-L-phenylalanine (pA-Phe). The recombinant cell may further express an N-monooxygenase, and the pA-Phe may be converted to pN-Phe.

Where the native metabolite is the chorismate, wherein the recombinant cell may be E. coli.

In the recombinant cell, the native metabolite may be the pA-Pyr, the one or more heterologous enzymes may comprise a heterologous N-monooxygenase, and the pA-Pyr may be converted to para-nitro-phenylpyruvate (pN-Pyr). The recombinant cell may further express an aminotransferase, and the pN-Pyr may be converted to the pN-Phe.

In the recombinant cell, the native metabolite may be the pA-Pyr, the one or more heterologous enzymes may comprise a heterologous aminotransferase, and the pA-Pyr may be converted to para-amino-L-phenylalanine (pA-Phe). The recombinant cell may further express an N-monooxygenase and the pA-Phe may be converted to pN-Phe.

The recombinant cell may be Pseudomonas fluorescens, the native metabolite may be the pN-Pyr, the heterologous enzymes may comprise a heterologous aminotransferase, and the pN-Pyr may be converted to the pN-Phe.

The recombinant cell may further comprise a target polypeptide and express a heterologous aminoacyl-tRNA synthetase and a transfer RNA. The pN-Phe may be incorporated into the target polypeptide in the recombinant cell without requiring exposure of the recombinant cell to exogenous pN-Phe. The target polypeptide having the pN-Phe may be at least 50% more immunogenic than the target polypeptide without the pN-Phe. The recombinant cell may not be exposed to exogenous pN-Phe.

A cell culture is also provided. The cell culture comprises the recombinant cell of the present invention in a culture medium. The culture medium may have glucose as the sole carbon source for the recombinant cell. The culture medium may not be supplemented with exogenous pN-Phe.

A method of producing para-nitro-L-phenylalanine (pN-Phe) by a recombinant cell is provided. The recombinant cell comprises one or more heterologous genes encoding one or more heterologous enzymes. The pN-Phe production method comprises expressing a native metabolite by the recombinant cell. The native metabolite is selected from the group consisting of chorismate, para-amino-phenylpyruvate (pA-Pyr) and para-nitro-phenylpyruvate (pN-Pyr). The pN-Phe production method further comprises expressing the one or more heterologous enzymes, and converting the native metabolite to the pN-Phe in the recombinant cell.

According to the pN-Phe production method, the native metabolite may be the chorismate, the one or more heterologous enzymes may comprise PapA, PapB and PapC. The method may further comprise expressing the PapA, the PapB and the PapC by the recombinant cell, and converting the chorismate to para-amino-phenylpyruvate (pA-Pyr) in the recombinant cell.

Where the native metabolite is the chorismate, the pN-Phe production method may further comprise expressing an N-monooxygenase by the recombinant cell and converting the pA-Pyr to para-nitro-phenylpyruvate (pN-Pyr) in the recombinant cell. The pN-Phe production method may further comprise expressing an aminotransferase by the recombinant cell, and converting the pN-Pyr to the pN-Phe in the recombinant cell.

Where the native metabolite is the chorismate, the pN-Phe production method may further comprise expressing an aminotransferase by the recombinant cell, and converting the pA-Pyr to para-amino-L-phenylalanine (pA-Phe) in the recombinant cell. The pN-Phe production method may further comprise expressing an N-monooxygenase by the recombinant cell, and converting the pA-Phe to the pN-Phe in the recombinant cell.

According to the pN-Phe production method, the native metabolite may be the chorismate, and the recombinant cell may be E. coli.

According to the pN-Phe production method, the native metabolite may be the pA-Pyr, and the one or more heterologous enzymes may comprise a heterologous N-monooxygenase. The pN-Phe production method may further comprise expressing the N-monooxygenase by the recombinant cell, and converting the pA-Pyr to para-nitro-phenylpyruvate (pN-Pyr) in the recombinant cell. The pN-Phe production method may further comprise expressing an aminotransferase by the recombinant cell, and converting the pN-Pyr to the pN-Phe in the recombinant cell.

According to the pN-Phe production method, the native metabolite may be the pA-Pyr, and the one or more heterologous enzymes may comprise a heterologous aminotransferase. The pN-Phe production method may further comprise expressing the aminotransferase by the recombinant cell, and converting the pA-Pyr to para-amino-L-phenylalanine (pA-Phe) in the recombinant cell. The pN-Phe production method may further comprise expressing an N-monooxygenase by the recombinant cell, and converting the pA-Phe to the pN-Phe in the recombinant cell.

According to the pN-Phe production method, the recombinant cell may be Pseudomonas fluorescens, the native metabolite may be pN-Pyr, and the heterologous enzymes comprise a heterologous aminotransferase. The pN-Phe production method may further comprise expressing the aminotransferase by the recombinant cell, and converting the pN-Pyr to the pN-Phe in the recombinant cell.

According to the pN-Phe production method, the recombinant cell may comprise a target polypeptide. The method may further comprise expressing a heterologous amino-acyl tRNA synthetase and a transfer RNA in the recombinant cell, and incorporating the pN-Phe into the target polypeptide in the recombinant cell without requiring exposure of the recombinant cell to exogenous pN-Phe. As a result, the target polypeptide having the pN-Phe would be produced.

A method of producing a target polypeptide having para-nitro-L-phenylalanine (pN-Phe) in the recombinant cell of the present invention is provided. The recombinant cell comprises the target polypeptide. The method comprises expressing a heterologous amino-acyl tRNA synthetase and a transfer RNA in the recombinant cell, and incorporating the pN-Phe into the target polypeptide in the recombinant cell without requiring exposure of the recombinant cell to exogenous pN-Phe. As a result, the target polypeptide having the pN-Phe is produced. The target polypeptide having the pN-Phe may be secreted by the recombinant cell. The target polypeptide having the pN-Phe may be on the surface of the recombinant cell. The target polypeptide having the pN-Phe may be at least 50% more immunogenic than the target polypeptide without the pN-Phe. The method may exclude exposing the recombinant cell to exogenous pN-Phe. The method may further comprise growing the recombinant cell in a culture medium having glucose as the sole carbon source for the recombinant cell. The culture medium may not be supplemented with exogenous pN-Phe.

### BRIEF DESCRIPTION OF THE DRAWINGS

FIG. 1 illustrates the engineered metabolic pathway used to achieve pN-Phe biosynthesis, with a focus on the heterologous enzymes used to convert chorismate to pN-Phe. Also shown and included in experiments described herein is one of many known strategies to deregulate aromatic amino acid biosynthesis for increased carbon flux to chorismate, which is the overexpression of a well-characterized mutant of the AroG protein (AroG*). AroG* is a feedback-resistant variant of the endogenous E. coli AroG enzyme that catalyzes the first committed step into the chorismate synthesis pathway. The heterologous pathway begins with conversion of chorismate to p-amino-phenylpyruvate (pA-Pyr) via three established enzymatic steps. This can be achieved in E. coli through heterologous expression of papABC genes from S. venezuelae or by obaDEF genes from P. fluorescens. The next step in this linear rendition of the pathway is oxidation of pA-Pyr to p-nitrophenylpyruvate (pN-Pyr) via the activity of a previously uncharacterized N-oxygenase, ObaC, which is also known as Obil. The last step is conversion of pN-Pyr to pN-Phe via amino transfer. In reality, the amino transfer step may be occurring on pA-Pyr to form pA-Phe first, with the N-oxygenase subsequently catalyzing conversion of pA-Phe to pN-Phe as the final step. The most probable native aminotransferase in E. coli that catalyzes this reaction is TyrB but it is likely that multiple aminotransferases can contribute to the formation of the amino acid.

FIG. 2 demonstrates the stability of the heterologous metabolites of the pathway in the presence of metabolically active E. coli in culture media including pA-Phe, pA-Pyr, pN-Phe, and pN-Pyr. The stability of the desired product pN-Phe is an important criterion to determine for success of this invention. Our results indicate that phenylalanine derivatives are fairly stable, whereas pyruvate derivatives are comparatively unstable. The latter instability may be due to endogenous aminotransferase activity.

FIG. 3 demonstrates the effect of supplementation of heterologous metabolites on cell doubling time as an indication of chemical toxicity. We added 1 mM of each intermediate to MG1655 cultures in LB media. We incubated cultures in 96-well plate format in a Spectramax i3x plate reader set to 37° C. with absorbance readings at 600 nm taken every 5 minutes for 12 hours to calculate doubling times and growth rate. Our results indicate that all compounds except for pN-Pyr exhibit minimal influence on cell growth rate.

FIG. 4 demonstrates that endogenous E. coli aminotransferases convert phenylpyruvate species pN-Pyr to its respective phenylalanine derivative, pN-Phe. We cultured E. coli MG1655 in LB media in shake flasks for 24 h, with supplementation of pN-Pyr at 250 μM. We chose this concentration due to solubility of pN-Pyr. We tracked conversion of this substrate using HPLC as previously described. FIGS. 5A-B demonstrate that exogenous supplementation of pure chemical standards or pA-Pyr or pA-Phe to the culture media of recombinant E. coli strains that express the ObaC N-oxygenase leads to production of pN-Phe. We collected samples over a 24 h period and metabolite concentration was measured via HPLC. Our results indicate that pN-Phe (FIG. 5A) is formed at modest yields (240±20 μM) by addition of pA-Phe (FIG. 5B), and poor yields by addition of pA-Pyr (31.2±1.5 μM). FIG. 6 shows an SDS-PAGE gel (protein gel electrophoresis) after overexpression and Nickel affinity purification of the ObaC protein. ObaC was successfully isolated in two forms, with an N-terminal hexahistidine tag and with an N-terminal beta-galactosidase fusion, C-terminal hexahistidine tag. Further experiments showed that our N-terminal hexahistidine tagged protein was nonfunctional.

FIG. 7 demonstrates first-time biochemical characterization of the purified ObaC protein. An in vitro assay was performed by mixing 10 μM purified B-gal-ObaC-(his₆ₓ) in a 1 mL reaction consisting of 25 mM phosphate buffer pH 7.0, 25 mM NaCl, 1.5% H202, 40% methanol with 2 mM pA-Phe or pA-Pyr. The reaction mixture was incubated for 3 h at 25° C., following which protein was removed by filtering through a 10 K Amicon centrifugal filter unit. The eluent was then analyzed via HPLC as previously described. Our results indicate that ObaC is active on both pA-Phe and pA-Pyr with yields of 46.1±2.7 μM pN-Phe and 38.7±0.9 M pN-Pyr respectively.

FIGS. 8A and 8B show liquid chromatography—mass spectrometry results confirming that the product created by the action of the purified ObaC protein on pA-Phe is pN-Phe using samples submitted to a Waters Acquity UPLC H-Class coupled to a single quadrupole mass detector 2 (SQD2) with an electrospray ionization source. A standard (FIG. 8A) demonstrates the elution time of pN-Phe (left panel) and the corresponding peak for pN-Phe (MW=210) is shown in the MS trace at (M+1)=211 (right panel). The sample from FIG. 7 was submitted and confirmed to contain a pN-Phe peak as well (FIG. 8B, left panel shows elution time, right panel shows molecular weight).

FIG. 9 demonstrates the biosynthesis of pN-Phe in LB medium supplemented with 1% glucose by recombinant E. coli strains that express the complete heterologous pathway genes and the aroG* gene. We combined individual pathway steps by co-transforming relevant plasmids and co-expressing the genes that they contain. Within the pCola vector, we cloned the pA-Phe synthesis pathway consisting of the papABC operon (kindly provided to us by Professor Ryan Mehl of Oregon State University). Within the pACYC vector, we cloned feedback resistant aroG*. Within the pZE vector, we cloned the N-oxygenase obaC. We also tested a few other combinations of expression cassettes that did not perform as well, or that expressed additional enzymes with non-significant effects on titer. We co-transformed plasmids into a strain of E. coli MG1655 (DE3) and performed production experiments in 5 mL volumes in a 14 mL culture tubes. We grew cultures at 30° C. in LB-glucose and induced at mid-exponential phase as previously described. These results demonstrate synthesis of nearly 200 μM pN-Phe after 24 hours of growth, which is comparable to the concentration exogenously supplied for nsAA incorporation experiments.

FIG. 10 demonstrates de novo biosynthesis of pN-Phe using M9-glucose medium. The best performing strain from the previously described experiment was cultured in 50 mL shake flask scale at 30° C. In addition, we cloned obaC into an operon with aroG* within the pACYC vector containing either a p15A or ColE1 origin of replication and tested this result. The results indicate synthesis of nearly 300 μM pN-Phe after 48 hours of growth in the top performing strain.

FIGS. 11A and 11B show mass spectrometry results authenticated using the UPLC-MS system previously described, confirming that the product biosynthesized by this E. coli strain in M9-glucose medium and isolated by chromatography is indeed pN-Phe. To test, an initial HPLC method was run using an Agilent 1100 series HPLC system with a Zorbax Eclipse Plus C18 column to purify the pN-Phe peak. A 100 μL injection was made with an initial mobile phase of solvent A:B=95:5 (solvent A, water, 0.1% trifluoroacetic acid; solvent B, acetonitrile, 0.1% trifluoroacetic acid) and maintained for 1 min. We then increased concentration of solvent B to 50% over a gradient for 24 min. Concentration was returned to 95% solvent A and equilibrated for 2 min. Flow rate was 1 mL/min and metabolites were tracked at 270 nm. During the run, the peak corresponding to pN-Phe was collected (FIG. 11A) and then submitted to UPLC-MS as previously described. A standard (FIG. 11B) demonstrates the elution time of pN-Phe and the corresponding peak for pN-Phe (MW=210) is shown in the MS peak at (M+1)=211. The de novo synthesis (pACYC-AroG+pCola-papABC+pZE-ObaC 24 h purified peak) sample (FIG. 11C) demonstrates similar elution time and MS peak at (M+1)=211.

FIG. 12 is an illustration that depicts how an NSAA incorporation assay is commonly implemented in live cells by a practitioner skilled in the art. A fluorescent reporter protein is chosen and its gene is modified to include an in-frame TAG sequence at the DNA level, resulting in an in-frame UAG codon at a designated location within the protein sequence. Upon co-expression with an engineered or natural aminoacyl-tRNA synthetase and tRNA pair, where the tRNA contains an anticodon that pairs with UAG, the amount of fluorescent protein produced per cell can be indicative of the level of NSAA incorporation. Thus, measurement of fluorescence normalized by culture optical density (FL/OD) provides a high-throughput measurement of NSAA incorporation, as long as the FL/OD measurement remains low in the absence of NSAA. High FL/OD in the absence of NSAA indicates likely undesired background incorporation of a standard amino acid, whereas low FL/OD in the absence of NSAA and high FL/OD in the presence of NSAA indicates a desired result.

FIG. 13 demonstrates the screening of aminoacyl-tRNA synthetases (AARSs) for selective pN-Phe incorporation. Previously engineered derivatives of the tyrosyl-tRNA synthetase from Methanocaldococcus jannaschii (MjTyrRS) were evaluated for their ability to incorporate pN-Phe and to not incorporate the pA-Phe that is formed as an intermediate in our heterologous pathway. GFP fluorescence at excitation and emission wavelengths of 488 and 528 nm, respectively and OD₆₀₀ were measured for each sample. For each synthetase (referred to as pAFRS, pNFRS, tetRS-C11, NapARS, and pCNFRS based on previous literature distinctions), we performed this screen in the presence and absence of externally supplied nsAA given the tendency of several synthetases to accept natural aromatic amino acids (primarily L-Tyr) that are always present in cells, which results in varying degrees of background GFP expression. Our results demonstrate identification of multiple synthetases with desired activity and specificity towards pN-Phe rather than pA-Phe.

FIGS. 14A and 14B demonstrate the effect of pN-Phe concentration on the NSAA incorporation level for different synthetases. The results, repeated months apart demonstrate that while the incorporation level of pN-Phe is dose-dependent, even at doses as low as 0.1 mM pN-Phe the incorporation level is elevated above what is seen for 2 mM pA-Phe addition. Given that biosynthetic titers observed have reached ˜0.3 mM in the extracellular media, these experiments strongly suggest that the coupling of biosynthesis and incorporation of pN-Phe will be feasible to a skilled practitioner in the art.

FIGS. 15A and 15B contain mass spectrometry results that are direct evidence that pN-Phe is indeed becoming incorporated within our target protein, a ubiquitin-fused GFP. To obtain purified protein sample, we co-transformed E. coli MG1655 (DE3) with a plasmid containing a previously published engineered derivative of the Methanococcus jannaschii TyrRS (TetRS-C11)³, a pZE-ObaC construct expressing the N-oxygenase ObaC and a ubiquitin fused GFP reporter containing an amber suppression codon encoded on a vanillate inducible promoter system (pCDF-Ub-UAG-GFP). Purified protein was analyzed using a Waters Acquity UPLC H-Class coupled to a Xevo G2-XS Quadrupole Time-of-Flight (QToF) Mass Spectrometer. Spectrum was analyzed from m/z 500 to 2000 and the spectra was deconvoluted using maximum entropy in MassLynx. The pN-Phe supplemented control sample (FIG. 15A) confirmed a mass of 37307 Da (theoretical MW) and the pA-Phe supplemented sample (FIG. 15B) confirmed mass of 37308 Da (theoretical MW). This demonstrates that by exogenous supplementation of pA-Phe, a pathway intermediate, to cells that co-express the ObaC monooxygenase and incorporation machinery, we can achieve biosynthesis and incorporation of pN-Phe at specific sites within proteins.

FIG. 16 is a protein sequence alignment of the aminoacyl-tRNA synthetases (AARSs), including MjTyrRS (SEQ ID NO: 12), pNFRS (SEQ ID NO: 13), pAFRS (SEQ ID NO: 14), NapARS (SEQ ID NO: 15), TetRS-C11 (SEQ ID NO: 16), and pCNFRS (SEQ ID NO: 17), used in described experiments. These sequences are visualized in JalView software after alignment performed using Clustal Omega (European Bioinformatics Institute).

FIGS. 17A-17F demonstrate the activity of putative N-monooxygenase enzymes identified through a sequence similarity network. (A) To start, a sequence similarity network was generated using 2,134 unique putative diiron monooxygenase sequences determined from NCBI BLAST of the N-oxygenases AurF, CmlI, AzoC and ObaC represented as 775 unique nodes. Edges are drawn between nodes with a minimum alignment score of 100. Unclustered nodes (56 total nodes representing 60 sequences) are not displayed. Previously characterized N-monooxygenases are highlighted in light gray (PvfB, PSAAO, CmlI, AzoC, AurF(RJ), AurF, AlmD, HamC and BezJ). Previously uncharacterized N-monooxygenases cloned and tested in this study are highlighted in white. ObaC is shown in dark gray. All other N-monooxygenases are shown in black. All the gene sequences for nodes highlighted in white were cloned and all but four expressed. However, only NO16 in cluster 13 had activity on pA-Phe via an in vivo screening assay. (B) shows in vivo screening of N-monooxygenase activity on pA-Phe via supplementation testing of 1 mM pA-Phe in cultures expressing N-monooxygenases via measurement of pN-Phe titer after 24 h fermentation in LB media at 37° C. (C) shows in vitro investigations of NO16 activity on pA-Phe and pA-Pyr. (D) shows a depiction of the pN-Phe synthesis plasmid setup for experiments of de novo pN-Phe synthesis in M9-glucose media in (E) and synthesis of pN-Phe in MOPS EZ Rich media in (F). Samples sizes are n=3 using biological replicates. Data shown are mean±standard deviation.

FIG. 18 shows an in vivo screen of N-monooxygenase enzymes from cluster 13 in FIG. 17A where 1 mM pA-Phe was supplemented and pN-Phe titers were measured at 3 h, 6 h, and 20 h. Samples sizes are n=3 using biological replicates. Data shown are mean±standard deviation.

FIG. 19 shows as sequence identity matrix comparing all N-monooxygenases tested in this work.

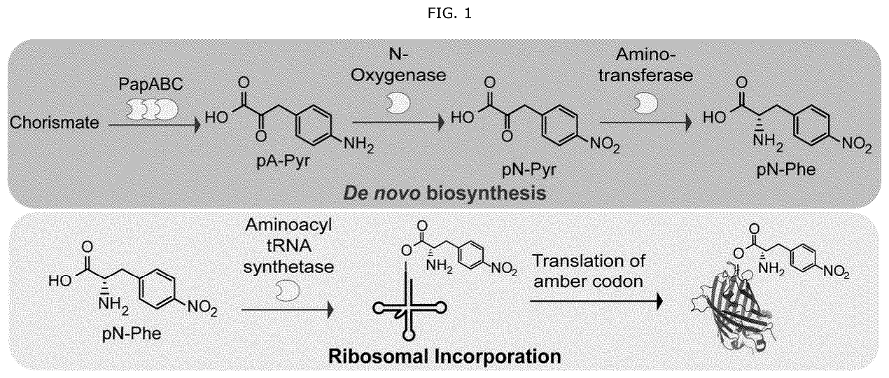

### DETAILED DESCRIPTION OF THE INVENTION

The present disclosure provides recombinant cells for producing para-nitro-L-phenylalanine (pN-Phe) from native metabolites and incorporating the pN-Phe into a target polypeptide in the cells without requiring exposure of the cells to exogenous pN-Phe. Also provided is a method of biosynthesizing para-nitro-L-phenylalanine (pN-Phe) in a cell, or biosynthesizing pN-Phe and incorporating the biosynthesized product within polypeptides in a cell. The method includes genetically modifying the cell to express heterologous pathway genes that result in the formation of pN-Phe from native metabolites. The method also includes producing a target polypeptide that includes pN-Phe substitution at an amino acid target location using an engineered aminoacyl-tRNA synthetase and transfer RNA pair corresponding to the non-standard amino acid, all without requiring supplementation of pN-Phe to culture media.

The invention is based on the discovery of a method for achieving the biosynthesis of pN-Phe by rerouting the metabolism of a microbe from native precursor metabolites to this non-native metabolic product. This is accomplished by introducing recombinant DNA from heterologous organisms into the desired microbial host strain through processes such as genetic transformation, so that the microbe will create non-native enzymes that catalyze biochemical reactions within the cell.

The inventors have discovered that the methods may be carried out in vivo, i.e. within a cell. Intermediate heterologous metabolite para-amino-phenylpyruvate (pA-Pyr) is formed from the natural metabolite chorismate as a result of the expression of three heterologous genes from organisms such as Streptomyces venezuelae (papABC) or Pseudomonas fluorescens (obaCDE). The intermediate heterologous metabolite pA-Pyr is converted to para-nitro-phenylpyruvate (pN-Pyr) by an N-monooxygenase related to the ObaC enzyme that is part of the obafluorin biosynthesis pathway found in Pseudomonas fluorescens. Alternatively, the intermediate heterologous metabolite pA-Pyr is modified by native cellular enzymes to form para-amino-L-phenylalanine (pA-Phe), in which case the N-monooxygenase then acts on pA-Phe to form the desired pN-Phe. The pN-Pyr is modified by one of several native E. coli aminotransferases, which are conserved across many bacteria, to form pN-Phe. The inventors have shown the results in the biosynthesis of pN-Phe by recombinant E. coli cells in a growth medium that contains glucose as the sole carbon source, thereby demonstrating achieving the standard expectation in the field for total biosynthesis.

The inventors have also discovered that the non-native or heterologous enzymes are made within a cell, i.e., in vivo. Certain cells, such as those of E. coli strains, naturally produce the enzymes needed to form the metabolite chorismate, which is the start of the heterologous metabolic pathway in the bacteria. Other cells may contain pA-Pyr as a native metabolite. Some cells, for example, P. fluorescens, contain pN-Pyr as a native metabolite. In all cases, man-made interventions in the form of gene additions or knockouts must be performed for cells to produce pN-Phe. In the case of certain cells such as E. coli, in addition to chorismate biosynthesis there must also be a native transaminase present that is capable of acting on pA-Pyr or pN-Pyr in order for the full pN-Phe biosynthesis pathway to function.

The inventors have further discovered a method of optimizing production of pN-Phe to increase metabolite titers such that its incorporation within protein antigens in live cells increases in feasibility. Reaction conditions are provided for making a target polypeptide including a non-standard amino acid substitution at an amino acid target location using an engineered amino-acyl tRNA synthetase and a transfer RNA as is known in the art. The amount of proteins having a desired non-standard amino acid is determined. Given the amount of protein produced, the reaction conditions and/or the amino-acyl tRNA synthetase and/or tRNA are altered and the amount of proteins having the desired non-standard amino acid is again determined. The process is repeated until the process is optimized for a desired yield of protein including desired NSAA. Exemplary reaction conditions which may be altered according to the present disclosure include changes of culture media, expression level of endogenous or heterologous genes, concentration of desired NSAA, or changes to the amino-acyl tRNA synthetase and/or tRNA including one or more mutations that may improve performance of the amino-acyl tRNA synthetase and/or tRNA. Such mutations may be made by methods known to those of skill in the art such as random mutagenesis approaches such as error-prone polymerase chain reaction (PCR) or directed approaches such as site-saturation mutagenesis or rational point mutagenesis.

The inventors have discovered a method of producing a modified protein that contains pN-Phe without the need for directly supplementing pN-Phe to microbial cultures by coupling components of the heterologous metabolic pathway and by using an amino-acyl tRNA synthetase that is engineered to incorporate pN-Phe in the target protein at an amino acid target location.

The term “recombinant cell” used herein refers to a cell that has been genetically modified to comprise at least one heterologous gene encoding at least one heterologous protein, for example, enzyme. The recombinant cell may express the heterologous protein. The protein may participate in a metabolic pathway for production of a desirable metabolite. Exemplary cells include prokaryotic cells and eukaryotic cells. Exemplary prokaryotic cells include bacteria, such as E. coli, such as genetically modified E. coli.

According to certain aspects, cells according to the present disclosure include prokaryotic cells and eukaryotic cells. Exemplary prokaryotic cells include bacteria. Microorganisms which may serve as host cells and which may be genetically modified to produce recombinant microorganisms as described herein may include one or members of the genera Shigella, Listeria, Salmonella, Clostridium, Escherichia, Rhodococcus, Pseudomonas, Bacillus, Lactobacillus, Saccharomyces, and Enterococcus. Particularly suitable microorganisms include bacteria and archaea. Exemplary microorganisms include Escherichia coli, Bacillus subtilis, and Saccharomyces cerevisiae. Exemplary eukaryotic cells include animal cells, such as human cells, plant cells, fungal cells and the like.

In addition to E. coli, other useful bacteria include but are not limited to Bacillus subtilis, Bacillus megaterium, Bifidobacterium bifidum, Caulobacter crescentus, Clostridium difficile, Chlamydia trachomatis, Corynebacterium glutamicum, Lactobacillus acidophilus, Lactococcus lactis, Listeria monocytogenes, Mycoplasma genitalium, Neisseria gonorrhoeae, Prochlorococcus marinus, Pseudomonas aeruginosa, Psuedomonas putida, Treponema pallidum, Salmonella enterica, Shigella dysenteriae, Streptomyces coelicolor, Synechococcus elongates, Vibrio natrigiens, and Zymomonas mobilis.

Exemplary genus and species of bacteria cells include Acetobacter aurantius, Acinetobacter bitumen, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella, Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

Exemplary genus and species of yeast cells include Saccharomyces, Saccharomyces cerevisiae, Torula, Saccharomyces boulardii, Schizosaccharomyces, Schizosaccharomyces pombe, Candida, Candida glabrata, Candida tropicalis, Yarrowia, Candida parapsilosis, Candida krusei, Saccharomyces pastorianus, Brettanomyces, Brettanomyces bruxellensis, Pichia, Pichia guilliermondii, Cryptococcus, Cryptococcus gattii, Torulaspora, Torulaspora delbrueckii, Zygosaccharomyces, Zygosaccharomyces bailii, Candida lusitaniae, Candida stellata, Geotrichum, Geotrichum candidum, Pichia pastoris, Kluyveromyces, Kluyveromyces marxianus, Candida dubliniensis, Kluyveromyces, Kluyveromyces lactis, Trichosporon, Trichosporon uvarum, Eremothecium, Eremothecium gossypii, Pichia stipitis, Candida milleri, Ogataea, Ogataea polymorpha, Candida oleophilia, Zygosaccharomyces rouxii, Candida albicans, Leucosporidium, Leucosporidium frigidum, Candida viswanathii, Candida blankii, Saccharaomyces telluris, Saccharomyces florentinus, Sporidiobolus, Sporidiobolus salmonicolor, Dekkera, Dekkera anomala, Lachancea, Lachancea kluyveri, Trichosporon, Trichosporon mycotoxinivorans, Rhodotorula, Rhodotorula rubra, Saccharomyces exiguus, Sporobolomyces koalae, and Trichosporon cutaneum, and other genus and species known to those of skill in the art.

Exemplary genus and species of fungal cells include Sac fungi, Basidiomycota, Zygomycota, Chtridiomycota, Basidiomycetes, Hyphomycetes, Glomeromycota, Microsporidia, Blastocladiomycota, and Neocallimastigomycota, and other genus and species known to those of skill in the art.

Exemplary eukaryotic cells include mammalian cells, plant cells, yeast cells and fungal cells.

The term “biosynthetic pathway”, also known as “metabolic pathway”, refers to a series of anabolic or catabolic biochemical reactions for conversion of one chemical species to another chemical species. When gene products (e.g., enzymes) act on the same substrate either in parallel or in series to produce the same product, or act on a metabolic intermediate (or “metabolite”) between the same substrate and metabolic final product, or produce the metabolic intermediate, the gene products belong to the same “metabolic pathway”.

The term “metabolite” used herein refers to a small molecule intermediate or end product of the set of enzymatic reactions which represent metabolism. Exemplary metabolites include chorismate, para-amino-phenylpyruvate (pA-Pyr), para-nitro-phenylpyruvate (pN-Pyr), para-amino-L-phenylalanine (pA-Phe), and para-nitro-L-phenylalanine (pN-Phe).

The terms “foreign”, “exogenous”, and “heterologous” are used herein interchangeably and refers to a molecule, for example, a polynucleotide (e.g., gene), a protein (e.g., enzyme), or a metabolite produced or expressed in a cell from a microorganism with genetic modification (i.e., recombinant cell) but not in a cell from the microorganism without any generic modifications.

The terms “natural”, “native”, “endogenous” and “homologous” are used interchangeably and refers to a molecule, for example, a polynucleotide (e.g., gene), a protein (e.g., enzyme), or a metabolite produced or expressed a cell from a microorganism without any generic modification.

The terms “production” and “expression” are used herein interchangeably and refer to transcription of a gene and/or translation of an mRNA transcript into a protein by a cell.

The term “feedstock” as used herein refers to a starting material, or a mixture of starting materials, supplied to a recombinant cell in a culture medium for production of a desirable molecule (e.g., metabolite). For example, a carbon source such as a biomass or a carbon compound derived from a biomass is a feedstock for a microorganism in a fermentation process or in other growth contexts, such as a live vaccine vector or immunotherapy. The feedstock may contain nutrients other than carbon sources.

The term “carbon source” as used herein refers to a substance suitable for use as a source of carbon, for a recombinant cell to grow. Carbon sources include, but are not limited to, glucose, biomass hydrolysates, starch, sucrose, cellulose, hemicellulose, xylose, lignin and monomer components of these substrates. Without being limitative, carbon sources may include various organic compounds in various forms including polymers, carbohydrates, acids, alcohols, aldehydes, ketones, amino acids and peptides. Examples of these include various monosaccharides, for example, glucose, dextrose (D-glucose), maltose, oligosaccharides, polysaccharides, saturated or unsaturated fatty acids, succinic acid, lactic acid, acetic acid, ethanol, rice bran, molasses, corn decomposition solution, cellulose decomposition solution, and mixtures of the foregoing.

The term “substrate” used herein refers to a compound that is converted to another compound by the action of one or more enzymes, or that is intended for such conversion. The term includes not only a single type of compound but also any combination of compounds, such as a solution, mixture or other substance containing at least one substrate or its derivative. Furthermore, the term “substrate” includes not only compounds that provide a carbon source suitable for use as a starting material such as sugar, derived from a biomass, but also intermediate and final product metabolites used in pathways associated with the metabolically manipulated microorganisms described in the present specification.

The terms “polynucleotide” and “nucleic acid” are used herein interchangeably and refer to an organic polymer comprising two or more monomers including nucleotides, nucleosides or their analogs, and include, but are not limited to, single-stranded or double-stranded sense or antisense deoxyribonucleic acid (DNA) of arbitrary length, and where appropriate, single-stranded or double-stranded sense or antisense ribonucleic acid (RNA) of arbitrary length, including siRNA.

The terms “protein” and “polypeptide” are used herein interchangeably and refer to an organic polymer composed of two or more amino acid monomers and/or analog and joined together by peptide bonds between the carboxyl and amino groups of adjacent amino acid residues.

The terms “amino acid” and “amino acid monomer” are used herein interchangeably and refer to a natural or synthetic amino acid, for example, glycine and both D- or L-optical isomers. The term “amino acid analog” as used herein refers to an amino acid wherein one or more individual atoms has been replaced with different atoms or different functional groups.

The term “non-standard amino acid (NSAA)” used herein refers to amino acids that are naturally encoded or found in the genetic code of any organism. Examples of NSAAs include pN-Phe and pA-Phe.

The present invention provides a recombinant cell for producing para-nitro-L-phenylalanine (pN-Phe). The recombinant cell comprises one or more heterologous genes encoding one or more heterologous enzymes. The recombinant cell expresses the one or more heterologous enzymes and a native metabolite. The native metabolite is selected from the group consisting of chorismate, para-amino-phenylpyruvate (pA-Pyr) and para-nitro-phenylpyruvate (pN-Pyr). In the recombinant cell, the native metabolite is converted to the pN-Phe.

According to the present invention, heterologous enzymes are involved in biosynthesis of pN-Phe in a recombinant cell. The enzymes may catalyze conversion of native metabolites to pN-Phe, directly or indirectly via intermediate metabolites. The intermediate metabolites may be selected from the group consisting of pA-Pyr, para-nitro-phenylpyruvate (pN-Pyr), para-amino-L-phenylalanine (pA-Phe) and a combination thereof. The heterologous enzymes may be selected from the group consisting of PapA, PapB and PapC, N-monooxygenases, aminotransferases, and a combination thereof. The heterologous genes may be introduced into the recombinant cell simultaneously or in sequence. A heterologous gene may be introduced into the recombinant cell permanently or transiently. The heterologous gene may be integrated into the genome of the recombinant cell.

The PapA may consist of the amino acid sequence of SEQ ID NO: 1, or an amino acid sequence at least 70%, 80%, 90%, 91%, 92%, 93%, 94%, 95%, 96%, 97%, 98% or 99% identical to the amino acid sequence of SEQ ID NO: 1 while maintaining the PapA enzymatic activity. The PapB may consist of the amino acid sequence of SEQ ID NO: 2, or an amino acid sequence at least 70%, 80%, 90%, 91%, 92%, 93%, 94%, 95%, 96%, 97%, 98% or 99% identical to the amino acid sequence of SEQ ID NO: 2 while maintaining the PapB enzymatic activity. The PapC may consist of the amino acid sequence of SEQ ID NO: 3, or an amino acid sequence at least 70%, 80%, 90%, 91%, 92%, 93%, 94%, 95%, 96%, 97%, 98% or 99% identical to the amino acid sequence of SEQ ID NO: 3 while maintaining the PapC enzymatic activity. The PapA, the PapB and the PapC may be PapABC from Streptomyces venezuelae. The PapA, the PapB and the PapC may be ObaDEF from Pseudomonas fluorescens.

The N-monooxygenase may be selected from the group consisting of ObaC, NO16and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens.

The aminotransferase may be from E. coli. The aminotransferase may be selected from the group consisting of TyrB, AspC, and IlvE.

Chorismate may be converted to pA-Pyr in the recombinant cell. The recombinant cell may comprise exogenous genes papA, papB and papC, for example, derived from Streptomyces venezuelae, encoding PapA, PapB and PapC, respectively. The PapA, PapB and PapC may catalyze the conversion of chorismate to pA-Pyr in the recombinant cell.

pA-Pyr may be converted to pN-Pyr in the recombinant cell. The recombinant cell may comprise an exogenous gene encoding an N-monooxygenase. The N-monooxygenase may catalyze the conversion of pA-Pyr to pN-Pyr.

pN-Pyr may be converted to pN-Phe in the recombinant cell. The recombinant cell may comprise an exogenous gene encoding an aminotransferase. The aminotransferase may catalyze the conversion of pN-Pyr to pN-Phe.

pA-Pyr may be converted to pA-Phe in the recombinant cell. The recombinant cell may comprise an exogenous gene encoding an aminotransferase. The aminotransferase may catalyze the conversion of pA-Pyr to pA-Phe.

pA-Phe may be converted to pN-Phe in the recombinant cell. The recombinant cell may comprise an exogenous gene encoding an N-monooxygenase. The N-monooxygenase may catalyze the conversion of pA-Phe to pN-Phe.

The recombinant cell may produce pN-Phe from glucose using an engineered metabolic pathway. The first heterologous steps in this pathway may be comprised of three or more exogenous genes having a function of biosynthesizing pA-Pyr from chorismate, to create a recombinant cell capable of producing pA-Pyr or pA-Phe from simple carbon sources under standard culturing conditions.

In preparation of the recombinant cell, at least one gene coding for improved carbon flux to the heterologous pathway may be included, which could be a gene knockout or gene overexpression. For example, this may be achieved by expression of a well-characterized feedback-resistant variant of the aroG gene from E. coli in the recombinant cell.

When the native metabolite is the chorismate, the recombinant cell may comprise heterologous genes encoding heterologous PapA, PapB and PapC, and the chorismate may be converted to para-amino-phenylpyruvate (pA-Pyr) in the recombinant cell. The recombinant cell may be E. coli. The recombinant cell expresses the heterologous PapA, PapB and PapC. The conversion of the chorismate to the pA-Pyr may be catalyzed by the PapA, PapB and PapC. The PapA may consist of an amino acid sequence at least 70%, 80%, 90%, 91%, 92%, 93%, 94%, 95%, 96%, 97%, 98%, 99% or 100% identical to the amino acid sequence of SEQ ID NO: 1. The PapB may consist of an amino acid sequence at least 70%, 80%, 90%, 91%, 92%, 93%, 94%, 95%, 96%, 97%, 98%, 99% or 100% identical to the amino acid sequence of SEQ ID NO: 2. The PapC may consist of an amino acid sequence at least 70%, 80%, 90%, 91%, 92%, 93%, 94%, 95%, 96%, 97%, 98%, 99% or 100% identical to the amino acid sequence of SEQ ID NO: 3. The PapA, the PapB and the PapC may be PapABC from Streptomyces venezuelae. The PapA, the PapB and the PapC may be ObaDEF from Pseudomonas fluorescens.

When the native metabolite is the chorismate, the recombinant cell may further express an N-monooxygenase, and the pA-Pyr may be converted to para-nitro-phenylpyruvate (pN-Pyr) in the recombinant cell. The recombinant cell may be E. coli. The N-monooxygenase may be native or heterologous. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The ObaC may be part of the obafluorin biosynthesis pathway found in Pseudomonas fluorescens. The recombinant cell may further express an aminotransferase, and the pN-Pyr is converted to the pN-Phe. The aminotransferase may be selected from the group consisting of TyrB, AspC, and IlvE.

When the native metabolite is the chorismate, the recombinant cell may further express an aminotransferase, and the pA-Pyr may be converted to para-amino-L-phenylalanine (pA-Phe). The recombinant cell may be E. coli. The aminotransferase may be native or heterologous. The aminotransferase may be selected from the group consisting of TyrB, AspC, and IlvE. The recombinant cell may further express an N-monooxygenase, and the pA-Phe may be converted to pN-Phe. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens.

When the native metabolite is the pA-Pyr, the recombinant cell may comprise a heterologous gene encoding a heterologous N-monooxygenase, and the pA-Pyr may be converted to para-nitro-phenylpyruvate (pN-Pyr). The N-monooxygenase may be native or heterologous. The N-monooxygenase may be selected from the group consisting of ObaC, ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens. The recombinant cell may further express an aminotransferase, and the pN-Pyr may be converted to the pN-Phe. The aminotransferase may be selected from the group consisting of TyrB, AspC, and IlvE.

When the native metabolite is pA-Pyr, the recombinant cell may comprise a heterologous gene encoding a heterologous aminotransferase, and the pA-Pyr may be converted to para-amino-L-phenylalanine (pA-Phe). The aminotransferase may be native or heterologous. The aminotransferase may be selected from the group consisting of TyrB, AspC, and IlvE. The recombinant cell may further express an N-monooxygenase, and the pA-Phe may be converted to pN-Phe. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47,or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens.

When the native metabolite is pN-Pyr, the recombinant cell may comprise a heterologous gene encoding a heterologous aminotransferase, and the pN-Pyr may be converted to the pN-Phe. The recombinant cell may be Pseudomonas fluorescens. The aminotransferase may be from E. coli. The aminotransferase may be selected from the group consisting of TyrB, AspC, and IlvE.

The recombinant cell of the present invention may further comprise a target polypeptide and express a heterologous aminoacyl-tRNA synthetase and a transfer RNA. The pN-Phe may be incorporated into the target polypeptide in the recombinant cell without requiring exposure of the recombinant cell to exogenous pN-Phe. The recombinant cell may secrete the target polypeptide having the pN-Phe. The target polypeptide having the pN-Phe may be on the surface of the recombinant cell. The target polypeptide may be immunogenic. The target polypeptide or the recombinant cell containing it may be administered to patients or to animals for immunization. The target polypeptide having the pN-Phe may be at least 30%, 40%, 50%, 60%, 70%, 80%, 90%, 95%, 100%, 200% or 300% more immunogenic than the target polypeptide without the pN-Phe. The recombinant cell may not be exposed to exogenous pN-Phe.

For each recombinant cell of the present invention, a cell culture is provided. The cell culture comprises the recombinant cell in a culture medium. The sole carbon source for the recombinant cell may be glucose, glycerol, or starch in the culture medium. In one embodiment, the sole carbon source for the recombinant cell is glucose in the culture medium. The culture medium may not be supplemented with exogenous pN-Phe.

A method of producing para-nitro-L-phenylalanine (pN-Phe) by a recombinant cell is provided. The recombinant cell comprises one or more heterologous genes encoding one or more heterologous enzymes. The pN-Phe production method comprises expressing a native metabolite by the recombinant cell. The method also comprises expressing the one or more heterologous enzymes, and converting the native metabolite to the pN-Phe in the recombinant cell. The native metabolite may be selected from the group consisting of chorismate, para-amino-phenylpyruvate (pA-Pyr) and para-nitro-phenylpyruvate (pN-Pyr).

When the native metabolite is the chorismate, the one or more heterologous enzymes may comprise PapA, PapB and PapC. The pN-Phe production method may further comprise expressing the PapA, the PapB and the PapC by the recombinant cell. The pN-Phe production method may further comprise converting the chorismate to para-amino-phenylpyruvate (pA-Pyr) in the recombinant cell. The recombinant cell may be E. coli. The recombinant cell may express the heterologous PapA, PapB and PapC. The conversion of the chorismate to the pA-Pyr may be catalyzed by the PapA, the PapB and the PapC. The PapA may consist of an amino acid sequence at least 70%, 80%, 90%, 91%, 92%, 93%, 94%, 95%, 96%, 97%, 98%, 99% or 100% identical to the amino acid sequence of SEQ ID NO: 1. The PapB may consist of an amino acid sequence at least 70%, 80%, 90%, 91%, 92%, 93%, 94%, 95%, 96%, 97%, 98%, 99% or 100% identical to the amino acid sequence of SEQ ID NO: 2. The PapC may consist of an amino acid sequence at least 70%, 80%, 90%, 91%, 92%, 93%, 94%, 95%, 96%, 97%, 98%, 99% or 100% identical to the amino acid sequence of SEQ ID NO: 3. The PapA, the PapB and the PapC may be PapABC from Streptomyces venezuelae. The PapA, the PapB and the PapC may be ObaDEF from Pseudomonas fluorescens.

When the native metabolite is the chorismate, the pN-Phe production method may further comprise expressing an N-monooxygenase by the recombinant cell, and converting the pA-Pyr to para-nitro-phenylpyruvate (pN-Pyr) in the recombinant cell. The recombinant cell may be E. coli. The N-monooxygenase may be native or heterologous. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens. The pN-Phe production method may further comprise expressing an aminotransferase, and the pN-Pyr may be converted to the pN-Phe. The aminotransferase may be selected from the group consisting of TyrB, AspC, and IlvE.

When the native metabolite is the chorismate, the pN-Phe production method may further comprise expressing an aminotransferase, and the pN-Pyr may be converted to the pN-Phe. The recombinant cell may be E. coli. The aminotransferase may be native or synthetic. The aminotransferase may be selected from the group consisting of TyrB, AspC, and IlvE. The pN-Phe production method may further comprise expressing an N-monooxygenase by the recombinant cell, and converting the pA-Phe to pN-Phe in the recombinant cell. The N-monooxygenase may be native or heterologous. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens.

When the native metabolite is the pA-Pyr, the recombinant cell may comprise a heterologous gene encoding a heterologous N-monooxygenase, and the pA-Pyr may be converted to para-nitro-phenylpyruvate (pN-Pyr). The N-monooxygenase may be native or heterologous. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens. The pN-Phe production method may further express an aminotransferase, and the pN-Pyr may be converted to the pN-Phe. The aminotransferase may be selected from the group consisting of TyrB, AspC, and IlvE.

When the native metabolite is the pA-Pyr, the recombinant cell may comprise a heterologous gene encoding a heterologous aminotransferase, and the pA-Pyr may be converted to para-amino-L-phenylalanine (pA-Phe). The aminotransferase may be native or heterologous. The aminotransferase may be selected from the group consisting of TyrB, AspC, and IlvE. The pN-Phe production method may further express an N-monooxygenase, and the pA-Phe may be converted to pN-Phe. The N-monooxygenase may be selected from the group consisting of ObaC, NO16 and NO22-NO29. The N-monooxygenase may consist of an amino acid sequence selected from the group consisting of SEQ ID NOS: 7, 33 and 39-47, or an amino acid sequence at least 50%, 55%, 60%, 70%, 80%, 90%, 91%, 92%, 93%, 94%, 95%, 96%, 97%, 98% or 99% identical to an amino acid sequence selected from the group consisting of SEQ ID NOS: 7, 33 and 39-47 while maintaining the enzymatic activity of the N-monooxygenase. In one embodiment, the N-monooxygenase may be NO16. The N-monooxygenase may consist of the amino acid sequence of SEQ ID NO: 33, or an amino acid sequence at least 50%, 55%, 60%, 70%, 80%, 90%, 91%, 92%, 93%, 94%, 95%, 96%, 97%, 98% or 99% identical to the amino acid sequence of SEQ ID NO: 33 while maintaining the enzymatic activity of NO16. The N-monooxygenase may be part of the obafluorin biosynthesis pathway found in Pseudomonas fluorescens.

When the native metabolite is the pN-Pyr, the recombinant cell may comprise a heterologous gene encoding a heterologous aminotransferase. The pN-Phe production method may further express the aminotransferase by the recombinant cell and the pN-Pyr may be converted to the pN-Phe. The recombinant cell may be Pseudomonas fluorescens. The aminotransferase may be from E. coli. The aminotransferase may be selected from the group consisting of TyrB, AspC, and IlvE.

The pN-Phe produced by the recombinant cell according to the present invention may incorporated into a target polypeptide in the recombinant cell. The target polypeptide may be immunogenic. Examples of the target polypeptide may include TNF-α, mRBP4, and C5a.

Basic to the present disclosure is the use of an amino-acyl tRNA synthetase/tRNA pair cognate to a nonstandard amino acid. Exemplary amino-acyl tRNA synthetase/tRNA pairs cognate to a nonstandard amino acid are known to those of skill in the art or may be designed for particular non-standard amino acids, as is known in the art or as described in Wang, Lei, and Peter G. Schultz. “Expanding the genetic code.” Angewandte chemie international edition 44.1 (2005): 34-66; Liu, Chang C., and Peter G. Schultz. “Adding new chemistries to the genetic code.” Annual review of biochemistry 79 (2010): 413-444; and Chin, Jason W. “Expanding and reprogramming the genetic code of cells and animals.” Annual review of biochemistry 83 (2014): 379-408. The aminoacyl-tRNA synthetase and transfer RNA pair corresponding to pN-Phe may be tetRS-C11, NapARS and pCNFRS paired to M. jannaschii tyrosyl tRNA^{CUA}.

The synthetase catalyzes a reaction that attaches the nonstandard amino acid to the correct tRNA. The amino-acyl tRNA then migrates to the ribosome. The ribosome adds the nonstandard amino acid where the tRNA anticodon corresponds to the reverse complement of the codon on the mRNA of the target protein to be translated. Only certain synthetases are capable of incorporating only pN-Phe rather than pA-Phe. This level of specificity is vital for the utilization of biosynthesized pN-Phe for introduction within protein sequences. The amino-acyl tRNA synthetase suitable for producing a target polypeptide having pN-Phe may be selected from the group consisting of tetRS-C11, NapARS and pCNFRS.

Where the recombinant cell comprises a target polypeptide, the pN-Phe production method may further comprise expressing a heterologous amino-acyl tRNA synthetase and a transfer RNA in the recombinant cell and incorporating the pN-Phe produced by the recombinant cell into the target polypeptide. The incorporation of the pN-Phe into the target polypeptide may take place in the recombinant cell. The incorporation of the pN-Phe into the target polypeptide in the recombinant cell may not require exposure of the recombinant cell to exogenous pN-Phe. The incorporation of the pN-Phe into the target polypeptide in the recombinant cell in the absence of exogenous pN-Phe. The method may exclude exposing the recombinant cell to exogenous pN-Phe. The method may further comprise growing the recombinant cell in a culture medium having a sole carbon source for the recombinant cell. The sole carbon source may be selected from the group consisting of glucose, glycerol, or starch. In one embodiment, the sole carbon source is glucose.

A method of producing a target polypeptide having pN-Phe in the recombinant cell of the present invention is provided. The recombinant cell comprises the target polypeptide. The method comprises expressing a heterologous amino-acyl tRNA synthetase and a transfer RNA in the recombinant cell. The method also comprises incorporating the pN-Phe produced by the recombinant cell into the target polypeptide in the recombinant cell. The incorporation of the pN-Phe into the target polypeptide may take place in the recombinant cell. The incorporation of the pN-Phe into the target polypeptide in the recombinant cell may not require exposure of the recombinant cell to exogenous pN-Phe. The incorporation of the pN-Phe into the target polypeptide in the recombinant cell in the absence of exogenous pN-Phe. The method may exclude exposing the recombinant cell to exogenous pN-Phe. The method may further comprise growing the recombinant cell in a culture medium having a sole carbon source for the recombinant cell. The sole carbon source may selected from the group consisting of glucose, glycerol, and starch. In one embodiment, the sole carbon source is glucose.

The target polypeptide having pN-Phe produced in the recombinant cell in accordance with the methods of the present invention may be secreted by the recombinant cell. The target polypeptide having the pN-Phe may be on the surface of the recombinant cell. The target polypeptide having the pN-Phe may be immunogenic. The immunogenicity of the target polypeptide having the pN-Phe may be at least 30%, 40%, 50%, 60%, 70%, 80%, 90%, 100%, 200%, 500% stronger than that of the target polypeptide without the pN-Phe.

### Example 1. Metabolite Supplementation Experiments for Pathway Development

When supplementing E. coli MG1655 (DE3) with pN-Phe, pA-Phe and pA-Pyr, no noticeable toxicity was detected and pN-Phe was fairly stable. Supplementation of E. coli MG1655 (DE3) expressing ObaC with 2 mM pA-Phe or 2 mM pA-Pyr resulted in yields of 240±20 μM and 31.2±1.5 μM, respectively.

### Example 2: De Novo Biosynthesis of pN-Phe

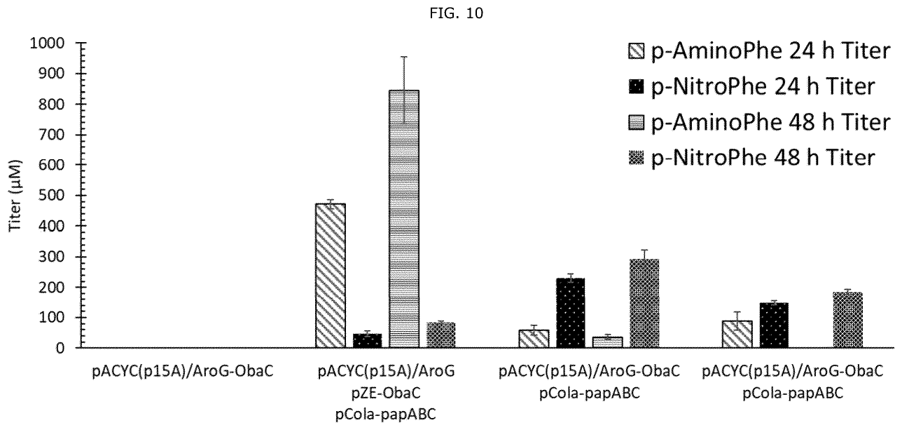

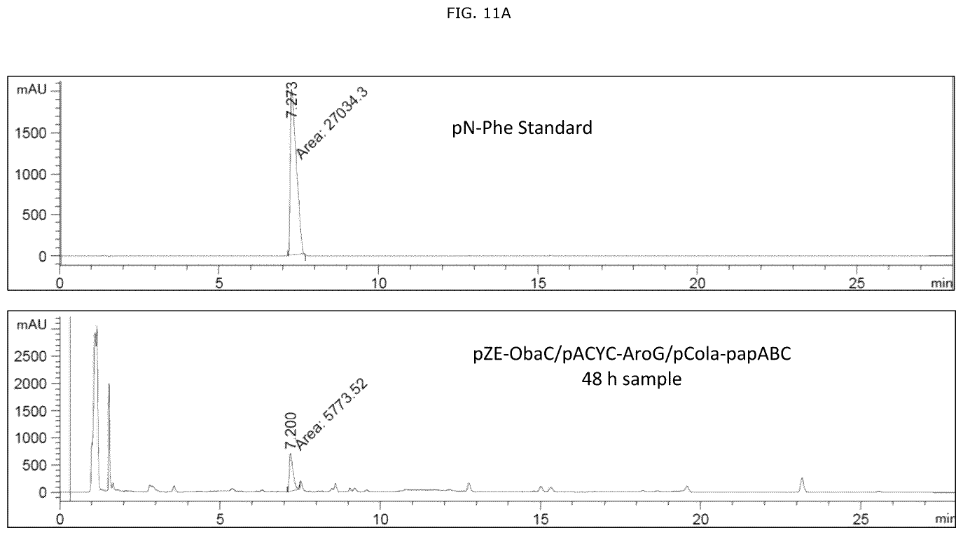

When E. coli MG1655 (DE3) was co-transformed with pCola-papABC, pACYC-AroG, and pZE-ObaC, and cultured in shake flasks using M9-glucose minimal media, 85±4 μM pN-Phe was produced (FIG. 10). This result was confirmed via UPLC-MS (FIGS. 11A-11C).

### Comparative Example 2

When E. coli MG1655 (DE3) was transformed with pACYC-AroG-ObaC, and cultured in shake flasks using M9-glucose minimal media, no pN-Phe was produced (FIG. 10).

### Example 3: Selective Ribosomal Protein Incorporation of pN-Phe

Expression of the AARSs tetRS-C11, NapARS, and pCNFRS in assays described in “NSAA incorporation assays” in Materials and Methods below results in selective incorporation of pN-Phe as opposed to pA-Phe as shown in FIG. 13 and FIGS. 14A-14B.

### Example 4: Biosynthesis of pN-Phe From pA-Phe Paired With Ribosomal Protein Incorporation

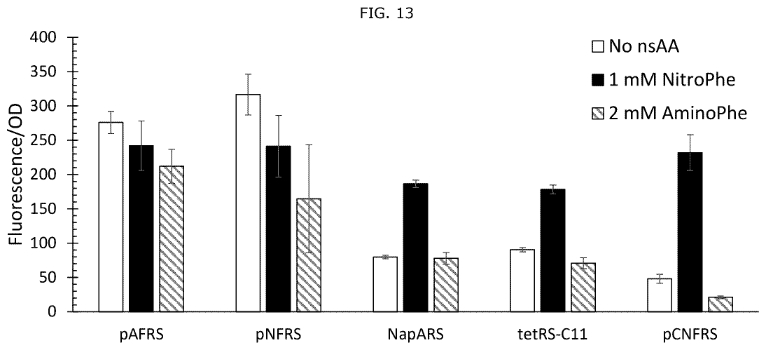

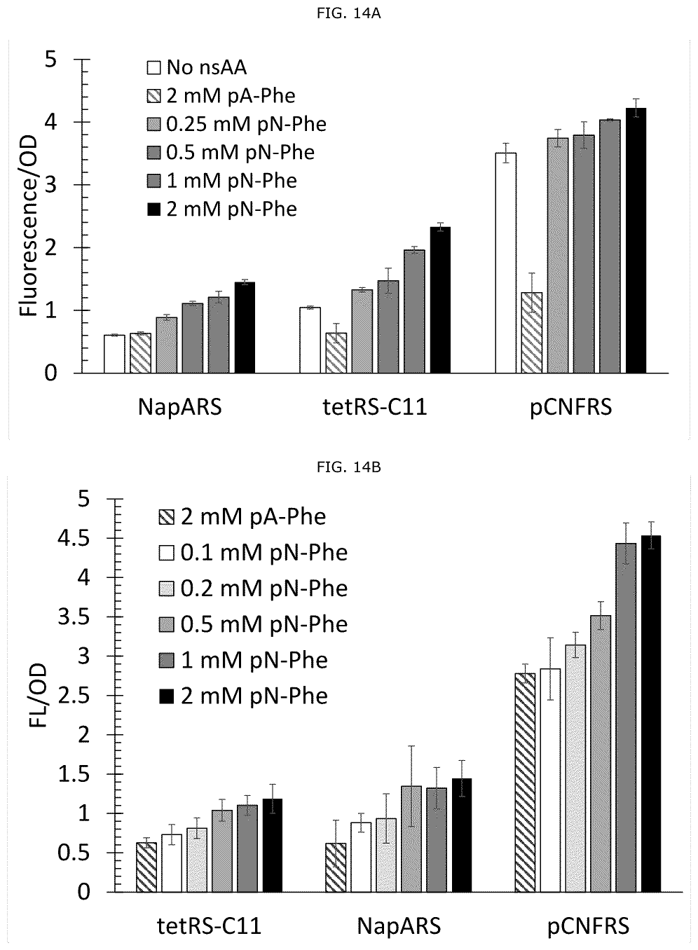

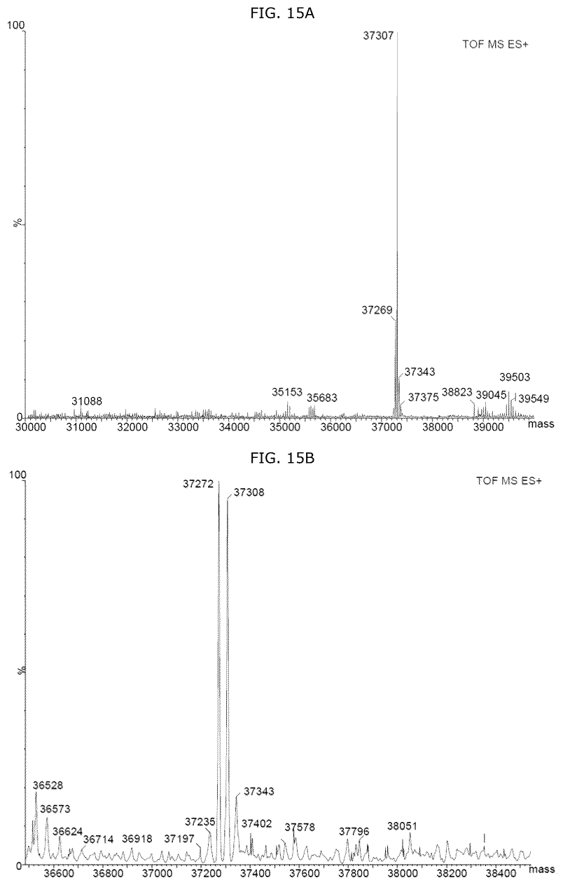

When E. coli MG1655 (DE3) was co-transformed with pEVOL-tetRS-C11, pZE-ObaC, and pCDF-Ub-UAG-GFP and reporter purification was performed as described in Materials and Methods, incorporation of pN-Phe was confirmed with supplementation of pN-Phe or pA-Phe (FIGS. 15A-15B).

### Materials and Methods

Strains and plasmids. E. coli strain and plasmids used are listed in Table 1. Molecular cloning and vector propagation were performed in DH5α. Polymerase chain reaction (PCR) based DNA replication was performed using KOD XTREME Hot Start Polymerase for plasmid backbones or using KOD Hot Start Polymerase otherwise.

Chemicals. The following compounds were purchased from MilliporeSigma: vanillic acid, hydrogen peroxide, kanamycin sulfate, chloramphenicol, carbenicillin disodium, dimethyl sulfoxide (DMSO), potassium phosphate dibasic, potassium phosphate monobasic, imidazole, glycerol, M9 salts, sodium dodecyl sulfate, lithium hydroxide, boric acid, HEPES, and KOD XTREME Hot Start and KOD Hot Start polymerases. pN-Phe and D-glucose were purchased from TCI America. pA-Phe, methanol, agarose, and ethanol were purchased from Alfa Aesar. pA-Pyr and pN-Pyr were purchased from abcr GmbH. Anhydrotetracycline (atc) and isopropyl β-D-1-thioglactopyranoside (IPTG) were purchased from Cayman Chemical. Acetonitrile, sodium chloride, Trace Elements A, LB Broth powder (Lennox), LB Agar powder (Lennox), were purchased from Fisher Chemical. L-Arabinose was purchased from VWR. Taq DNA ligase was purchased from GoldBio. Phusion DNA polymerase and T5 exonuclease were purchased from NEB. Sybr Safe DNA gel stain was purchased from Invitrogen.

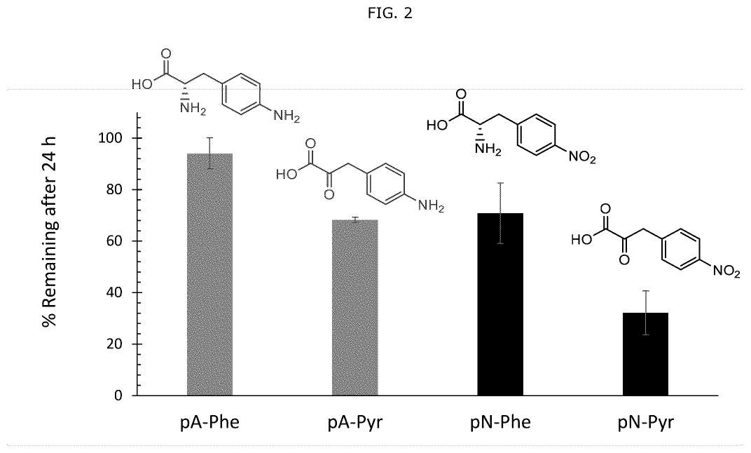

Culture conditions. Cultures for general culturing, for experiments in FIGS. 2-5B, 9, 13-16 and ObaC protein overexpression were grown in LB-Lennox medium (LBL: 10 g/L bacto tryptone, 5 g/L sodium chloride, 5 g/L yeast extract). Cultures to demonstrate de novo pN-Phe synthesis were grown in either LB-Lennox-glucose medium (LBL with 1% glucose (wt/vol)) or M9-glucose minimal media (200 mM MgSO₄, 10 mM CaCl₂, 8.5 g/L Na₂HPO₄·2H₂O, 3 g/L KH₂PO₄ 1 g/L NH₄Cl, 0.5 g NaCl, trace elements A (1000× dilution), 1.5% glucose).

For stability testing, a culture of E. coli K12 MG1655 (DE3) was inoculated from a frozen stock and grown to confluence overnight in 5 mL of LB media. Confluent overnight cultures were then used to inoculate experimental cultures in 300 μL volumes in a 96-deep-well plate (Thermo Scientific™ 260251) at 100× dilution. Cultures were supplemented with 0.5 mM of heterologous metabolites (pA-Phe, pA-Pyr, pN-Pyr, pN-Phe), with pN-Pyr requiring an addition of 15 μL of DMSO (˜5% final concentration) for solubility. Cultures were incubated at 37° C. with shaking at 1000 RPM and an orbital radius of 3 mm. Compounds were quantified from the extracellular broth over a 24 h period using HPLC.

For toxicity testing, cultures were similarly prepared with confluent overnight cultures of MG1655 (DE3) used to inoculate experimental cultures at 100× dilution in 200 μL volumes in a Greiner clear bottom 96 well plate (Greiner 655090) in LB media. Cultures were supplemented with 1 mM of heterologous metabolite and 5% DMSO for metabolite solubility and grown for 24 h in a Spectramax i3x plate reader with medium plate shaking at 37° C. with absorbance readings at 600 nm taken every 5 min to calculate doubling time and growth rate.

For supplementation testing, strains transformed with plasmids expressing pathway genes were prepared with inoculation of 300 μL volumes in a 96-deep-well plate with appropriate antibiotic added to maintain plasmids (34 μg/mL chloramphenicol (Cm), 50 μg/mL kanamycin (Kan), 50 μg/mL carbenicillin (Carb), or 95 μg/mL streptomycin (Str)). Cultures were incubated at 37° C. with shaking at 1000 RPM and an orbital radius of 3 mm until an OD600 of 0.5-0.8 was reached. At this point, the pathway plasmids were fully induced with addition of corresponding inducer (1 mM IPTG, 1 mM vanillate, or 0.2 nM anhydrotetracycline), and metabolite of interest was supplemented at this time. Cultures were incubated over 24 h at 37° C. with sampling and metabolite concentration measured via supernatant sampling and submission to HPLC.

For pN-Phe synthesis testing in LB-glucose media, overnight cultures from frozen stocks were grown with 1% glucose added. The following day, cultures of MG1655 (DE3) strains expressing pathway genes on different plasmid vectors were inoculated using confluent overnight cultures at a 100× dilution in 5 mL of LB-glucose with appropriate antibiotics added within 14 mL culture tubes. Cultures were grown at 30° C. at 250 RPM and expression vectors were fully induced at OD600 0.5-0.8. Synthesis of metabolites was quantified via supernatant sampling over 24 h and analysis by HPLC.

For de novo pN-Phe synthesis testing in M9-glucose minimal media, cultures were similarly inoculated with overnight culture in LB-glucose media. Cultures were inoculated at 100x dilution from confluent overnight culture in 50 mL M9-glucose media in 250 ml baffled shake flasks at 30° C. at 250 RPM. Expression vectors were fully induced at OD₆₀₀ 0.5-0.8. Synthesis of metabolites was quantified via supernatant sampling over 48 h and analysis by HPLC.

Overexpression and purification of ObaC. A strain of E. coli BL21 (DE3) harboring a pZE-ObaC plasmid with a hexahistidine tag at either the N-terminus or C-terminus with a beta-galactosidase fusion will be inoculated from frozen stocks and grown to confluence overnight in 5 mL LB containing kanamycin. Confluent cultures were used to inoculate 400 mL of experimental culture of LB supplemented with kanamycin. The culture was incubated at 37° C. until an OD₆₀₀ of 0.5-0.8 was reached while in a shaking incubator at 250 RPM.

ObaC expression was induced by addition of anhydrotetracycline (0.2 nM) and cultures were incubated at 30° C. for 5 h. Cultures were then grown at 20° C. for an additional 18 h. Cells were centrifuged using an Avanti J-15R refrigerated Beckman Coulter centrifuge at 4° C. at 4,000 g for 15 min. Supernatant was then aspirated and pellets were resuspended in 8 mL of lysis buffer (25 mM HEPES, 10 mM imidazole, 300 mM NaCl, 10% glycerol, pH 7.4) and disrupted via sonication using a QSonica Q125 sonicator with cycles of 5 s at 75% amplitude and 10 s off for 5 minutes. The lysate was distributed into microcentrifuge tubes and centrifuged for 1 h at 18,213 g at 4° C. The protein-containing supernatant was then removed and loaded into a HisTrap Ni-NTA column using an ÄKTA Pure GE FPLC system. Protein was washed with 3 column volumes (CV) at 60 mM imidazole and 4 CV at 90 mM imidazole. ObaC was eluted in 250 mM imidazole in 1.5 mL fractions. Selected fractions were run on an SDS-PAGE gel to identify protein containing fractions and confirm their size. The ObaC containing fractions were combined applied to an Amicon column (10 kDa MWCO) and diluted ˜1,000× into a 20 mM Tris pH 8.0, 5% glycerol buffer.

In vitro ObaC activity assay. Reactions were performed in 1 mL volumes consisting of 25 mM phosphate buffer pH 7.0, 25 mM NaCl, 1.5% H₂O₂, and 40% methanol with 1 mM pA-Phe or pA-Pyr. The reaction mixture was incubated for 6 h at 25° C., following which protein was removed by filtering through a 10 K Amicon centrifugal filter unit. The eluent was then analyzed by HPLC, and pN-Phe or pN-Pyr production was further confirmed via UPLC-MS.

NSAA incorporation assays. MjTyrRS derivatives were cloned within pEVOL plasmids and transformed into E. coli MG1655 (DE3) strain with a pZE plasmid expressing a reporter protein fusion consisting of a ubiquitin domain, followed by an in-frame amber suppression codon, followed by GFP (pZE-Ub-UAG-GFP). These transformed strains were cultured at 37° C. in 300 μL LB broth in deep 96-well plates with 0.2% (wt/v) L-arabinose, 1 mM NSAA, 34 μg/mL chloramphenicol, and 50 μg/mL kanamycin with shaking at 1000 RPM and an orbital radius of 3 mm. At mid-exponential growth (OD˜0.5), 0.2 nM ATC was added to induce transcription of mRNA that requires UAG suppression to form full-length GFP. Cultures were grown for 18 h at 37° C. before pelleting them via centrifugation. To eliminate possible fluorescence or absorbance via free NSAAs in culture media, cultures were washed in PBS buffer before quantification of both GFP fluorescence at excitation and emission wavelengths of 488 and 528 nm, respectively, and OD₆₀₀. For each synthetase, we performed this screen in the presence and absence of externally supplied nsAA.

Reporter purification. E. coli MG1655 (DE3) co-transformed with a plasmid containing a previously published engineered derivative of the Methanococcus jannaschii TyrRS (TetRS-C11)³, a pZE-ObaC construct expressing the N-oxygenase ObaC and a ubiquitin fused GFP reporter containing an amber suppression codon encoded on a vanillate inducible promoter system (pCDF-Ub-UAG-GFP). These strains were cultured at 37° C. in 50 mL of LB broth in 250 mL baffled shake flasks with 0.2% (wt/v) arabinose, 1 mM nsAA, 25.5 μg/mL chloramphenicol, 37.5 μg/mL kanamycin, 71.3 μL streptomycin, and 0.2 nM ATC in a shaking incubator at 250 RPM. At an OD600 of 0.5-0.8, 1 mM vanillate was added to induce transcription of mRNA that requires UAG suppression to form full-length GFP. Cultures were then grown at 37° C. for an additional 18 h. Reporter was purified in the presence of pN-Phe or pA-Phe. The reporter protein was then lysed and purified using FPLC with a His-Trap column as previously described. The protein sample was then concentrated using a 10 kDa MWC Amincon column and then diluted 10:1 in 10 mM ammonium acetate buffer and spun down to 1 mL samples three times. Then, the sample was diluted 10:1 in 2.5 mM ammonium acetate buffer and spun down to 1 mL samples three times. Protein in 2.5 mM ammonium acetate buffer was then submitted for whole-protein LC-MS.

HPLC Analysis. Metabolites of interest were quantified via high-performance liquid chromatography (HPLC) using an Agilent 1260 infinity model equipped with a Zorbax Eclipse XDB-C18 column. To quantify amine containing metabolites, an initial mobile phase of solvent A/B=100/0 was used (solvent A, 20 μM potassium phosphate, pH 7.0; solvent B, acetonitrile/water at 50/50) and maintained for 7 min. A gradient elution was performed (A/B) with: gradient from 100/0 to 50/50 for 7-17 min, gradient from 50/50 to 100/00 for 17-18 min, equilibration at 100/0 for 18-22 min. A flow rate of 0.5 mL min⁻¹ was maintained and absorption was monitored at 210 nm. To quantify nitro-group containing metabolites, we used an initial mobile phase of solvent A/B=100/0 (solvent A, water, 0.1% trifluoroacetic acid; solvent B, acetonitrile, 0.1% trifluoroacetic acid) and maintained for 5 min. A gradient elution (A/B) was then performed with: gradient from 100/0 to 95/5 over 5-7 min, gradient from 95/5 to 90/10 over 7-10 min, gradient from 90/10 to 80/20 over 10-16 min, gradient from 80/20 to 70/30 over 16-19 min, gradient from 70/30 to 0/100 over 19-21 min, 0/100 over 21-23 min, gradient from 0/100 to 100/0 over 23-24 min, and equilibration at 100/0 over 24-25 min. The nitro product quantifying method used flow rate of 0.5 mL min 1 and monitored absorption at 210 nm.

Mass Spectrometry. Mass spectrometry (MS) measurements for small molecule metabolites were submitted to a Waters Acquity UPLC H-Class coupled to a single quadrupole mass detector 2 (SQD2) with an electrospray ionization source. Metabolite compounds were analyzed using a Waters Cortecs UPLC C18 column with an initial mobile phase of solvent A/B=95/5 (solvent A, water, 0.1% formic acid; solvent B, acetonitrile, 0.1% formic acid) with a gradient elution from (A/B) 95/5 to 5/95 over 5 min. Flow rate was maintained at 0.5 mL min⁻¹. For samples collected from E. coli growth cultures, an initial submission to an Agilent 1100 series HPLC system with a Zorbax Eclipse Plus C18 column was used to collect pN-Phe elution peaks for enhanced MS resolution. A 100 ul injection was made with an initial mobile phase of solvent A/B=95/5 (solvent A, water, 0.1% trifluoroacetic acid; solvent B, acetonitrile, 0.1% trifluoroacetic acid) and maintained for 1 min. A gradient elution was then performed (A/B) with: gradient from 95/5 to 50/50 over 1-24 min, gradient from 50/50 to 95/5 over 24-25 min, equilibration at 95/5 for 25-27 min. Flow rate was 1 mL/min and metabolites were tracked at 270 nm. pN-Phe elution was identified at 7.20 min using a chemical standard and this peak was collected for submission to UPLC-MS.

For intact protein MS measurements, samples were submitted to a Waters Acquity UPLC H-Class coupled to a Xevo G2-XS Quadrupole Time-of-Flight (QToF) Mass Spectrometer. Protein sample was injected with an initial mobile phase of solvent A/B=85/15 (solvent A, water, 0.1% formic acid; solvent B, acetonitrile, 0.1% formic acid) held at 85/15 for 1 minute followed by a gradient elution from (A/B) 85/5 to 5/95 over 5 min. Flow rate was maintained at 0.5 mL min 1. Spectrum was analyzed from m/z 500 to 2000 and the spectra was deconvoluted using maximum entropy in MassLynx.

### Experimental Results

The stability of the desired product pN-Phe is an important criterion to determine for success of this invention. Chemicals were purchased off-the-shelf for pA-Pyr, pA-Phe, pN-Pyr, and pN-Phe and then added separately at a concentration of 1 mM to the wild-type E. coli K-12 MG1655 strain at mid-exponential phase during aerobic culturing in lysogeny broth (LB medium). Cultures were prepared at volumes of 300 μL in a 96-deep-well plate and incubated at 37° C. with shaking at 1000 rpm and an orbital radius of 3 mm. Compounds were quantified from the extracellular culture broth 24 hours after chemical supplementation via high-performance liquid chromatography (HPLC) using an Agilent 1260infinity model equipped with a Zorbax Eclipse XDB-C18 column. To quantify amine containing metabolites, we used an initial mobile phase of solvent A:B=100:0 (solvent A, 20 μM potassium phosphate, pH 7.0; solvent B, acetonitrile: water at 1:1 ratio) and maintained for 7 min. We then increased concentration of solvent B to 50% over a gradient for 10 min and then maintained for 1 min. Concentration was returned to 100% solvent A and equilibrated for 1 min. We used a flow rate of 0.5 mL min-1 and monitored absorption at 210 nm. To quantify nitro-group containing metabolites, we used an initial mobile phase of solvent A:B=100:0 (solvent A, water, 0.1% trifluoroacetic acid; solvent B, acetonitrile, 0.1% trifluoroacetic acid) and maintained for 5 min. We then increased concentration of solvent B 5% over a gradient for 2 min, then 10% over a gradient for 3 min, then 20% over a gradient for 6 min, followed by 30% over a gradient for 3 min, followed by 100% over a gradient for 2 min and then held at 100% B for 2 min. Concentration was returned to 100% solvent A and equilibrated for 1 min. Our results (FIG. 2) indicate that phenylalanine derivatives are fairly stable, whereas pyruvate derivatives are comparatively unstable. The latter instability may be due to endogenous aminotransferase activity.

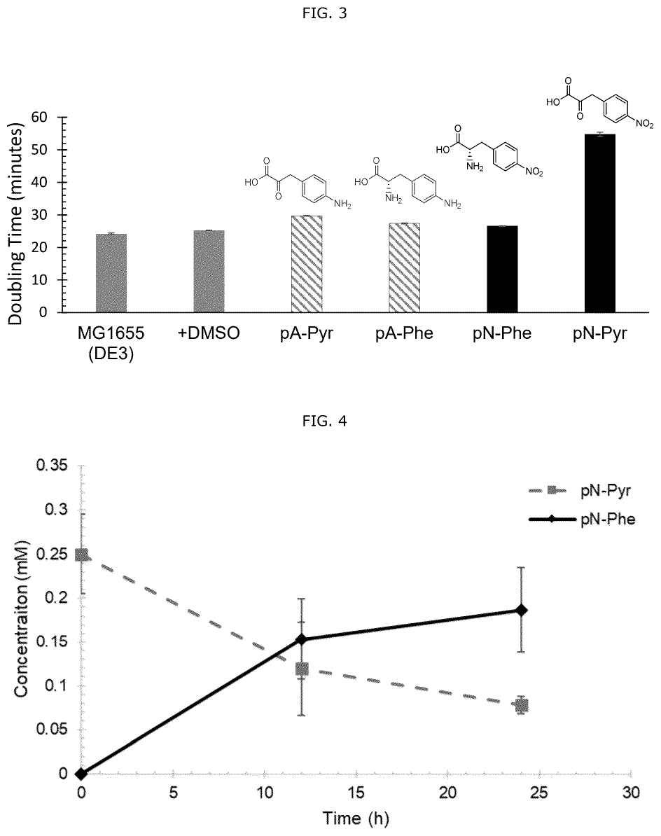

To determine the chemical toxicity of heterologous metabolites pA-Phe, pA-Pyr, pN-Phe, and pN-Pyr, we supplemented heterologous metabolites to cell culture and measured the effect on cell doubling time (FIG. 3). We added 1 mM of each intermediate to MG1655cultures in LB media. We incubated cultures in 96-well plate format in a Spectramax i3x plate reader set to 37° C. with absorbance readings at 600 nm taken every 5 minutes for 12 hours to calculate doubling times and growth rate. Our results indicate that all compounds except for pN-Pyr exhibit minimal influence on cell growth rate.

We then demonstrated that endogenous E. coli aminotransferases convert phenylpyruvate species pN-Pyr to its respective phenylalanine derivative, pN-Phe (FIG. 4). We cultured E. coli MG1655 in LB media in shake flasks for 24 h, with supplementation of pN-Pyr at 250 μM. We chose this concentration due to solubility of pN-Pyr. We tracked conversion of this substrate using HPLC as previously described.

Using exogenous supplementation recombinant E. coli strains that express the ObaC N-oxygenase, we then confirmed metabolic conversion using recombinant strains of pure chemical standards or pA-Pyr or pA-Phe to production of pN-Phe. We cloned the obaC gene into a pZE vector with a 6x C-terminal histidine tag. We transformed this plasmid into an MG1655 strain, grew cultures in LB medium to OD˜0.5, and added 0.2 nM ATC inducer and 1 mM pA-Pyr or pA-Phe. Although it had not been previously investigated as a substrate of ObaC, we tested pA-Phe because of the possibility that endogenous aminotransferases are more active than ObaC on pA-Pyr. We collected samples over a 24 h period and performed HPLC analysis as previously described. Our results indicate that pN-Phe is formed at modest yields (240±20 μM) by addition of pA-Phe (FIG. 5A), and poor yields (31.2±1.5 μM) by addition of pA-Pyr (FIG. 5B).

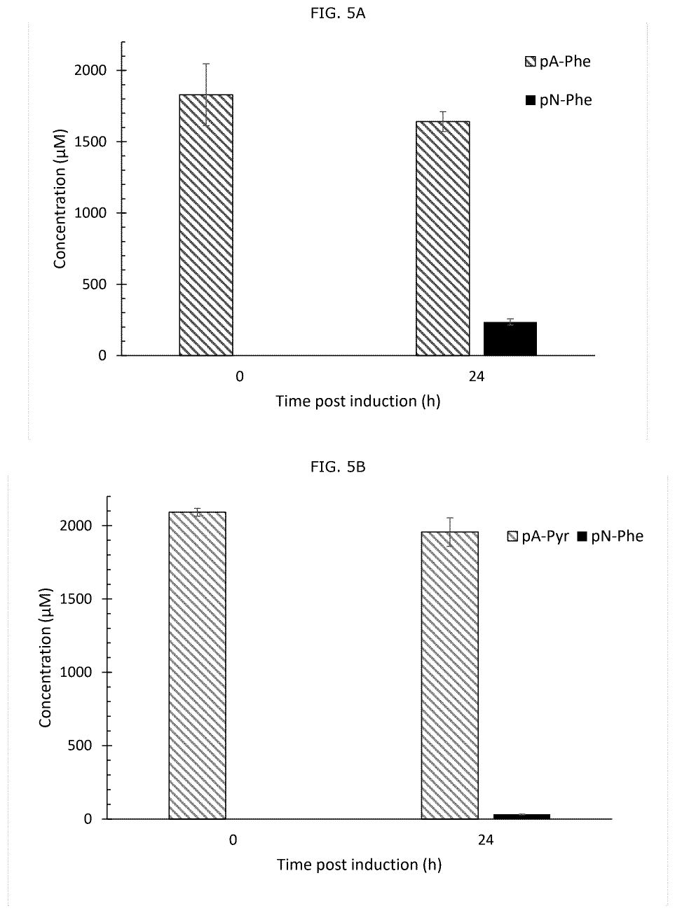

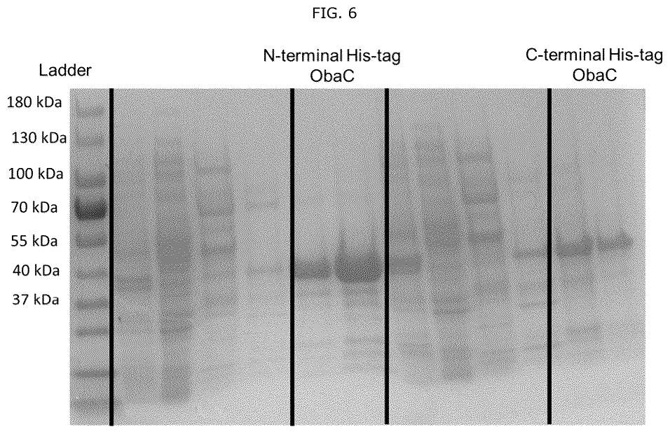

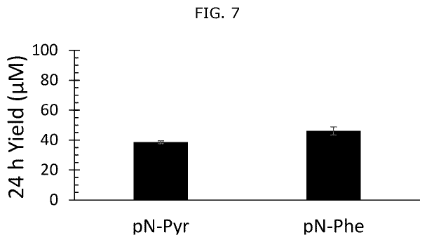

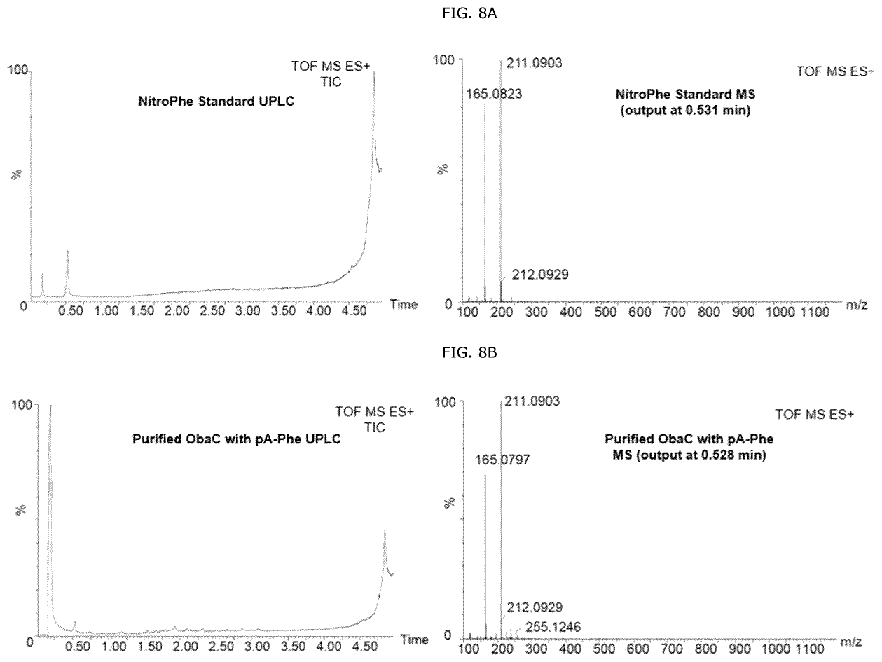

We successfully isolated in two forms, with an N-terminal hexahistidine tag and with an N-terminal beta-galactosidase fusion, C-terminal hexahistidine tag after overexpression and Nickel affinity purification of the ObaC proteins as demonstrated using SDS-PAGE protein gel electrophoresis (FIG. 6). Further experiments showed that our N-terminal hexahistidine tagged protein was nonfunctional. We characterized the N-terminal beta-galactosidase fusion, C-terminal hexahistidine tag ObaC protein using an in vitro assay performed by mixing 10 μM purified B-gal-ObaC-(his6x) in a 1 mL reaction consisting of 25 mM phosphate buffer pH 7.0, 25 mM NaCl, 1.5% H2O2, 40% methanol with 2 mM pA-Phe or pA-Pyr. The reaction mixture was incubated for 3 h at 25° C., following which protein was removed by filtering through a 10 K Amicon centrifugal filter unit. The eluent was then analyzed via HPLC as previously described. Our results (FIG. 7) indicate that ObaC is active on both pA-Phe and pA-Pyr with yields of 46.1±2.7 μM pN-Phe and 38.7±0.9 μM pN-Pyr respectively. The result for pA-Phe conversion was confirmed by UPLC-MS (FIGS. 8A-8B) samples submitted to a Waters Acquity UPLC H-Class coupled to a single quadrupole mass detector 2 (SQD2) with an electrospray ionization source. A standard (FIG. 8A) demonstrates the elution time of pN-Phe and the corresponding peak for pN-Phe (MW=210) is shown in the MS peak at (M+1)=211. In vitro ObaC reaction sample (FIG. 8B) demonstrates similar elution time and MS peak at (M+1)=211.

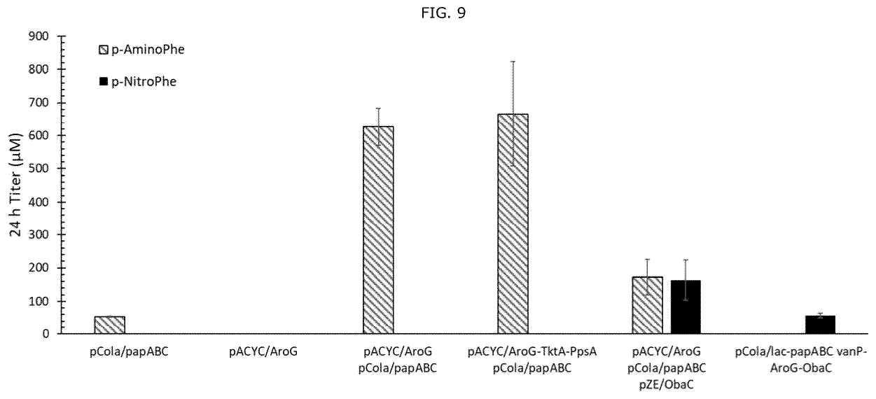

We then performed initial demonstration of the biosynthesis of pN-Phe in LB medium supplemented with 1% glucose by recombinant E. coli strains that express the complete heterologous pathway genes and the aroG* gene. We combined individual pathway steps by co-transforming relevant plasmids and co-expressing the genes that they contain. Within the pCola vector, we cloned the pA-Phe synthesis pathway consisting of the papABC operon (kindly provided to us by Professor Ryan Mehl of Oregon State University). Within the pACYC vector, we cloned feedback resistant aroG*. Within the pZE vector, we cloned the N-oxygenase obaC. We also tested a few other combinations of expression cassettes that did not perform as well, or that expressed additional enzymes with non-significant effects on titer. We co-transformed plasmids into a strain of E. coli MG1655 (DE3) and performed production experiments in 5 mL volumes in a 14 mL culture tubes. We grew cultures at 30° C. in LB-glucose and induced at mid-exponential phase as previously described. These results demonstrate synthesis of nearly 200 μM pN-Phe after 24 hours of growth (FIG. 9), which is comparable to the concentration exogenously supplied for nsAA incorporation experiments.

We then demonstrated de novo biosynthesis of pN-Phe using M9-glucose medium (FIG. 10). The best performing strain from the previously described experiment was cultured in 50 mL shake flask scale at 30° C. In addition, we cloned obaC into an operon with aroG* within the pACYC vector containing either a p15A or ColE1 origin of replication and tested this result. The results indicate synthesis of nearly 300 μM pN-Phe after 48 hours of growth in the top performing strain. We then used UPLC-MS to confirm the result of the previous best performing strain, confirming that the product biosynthesized by this E. coli strain in M9-glucose medium and isolated by chromatography is indeed pN-Phe. To test, an initial HPLC method was run using an Agilent 1100 series HPLC system with a Zorbax Eclipse Plus C18 column to purify the pN-Phe peak. A 100 uL injection was made with an initial mobile phase of solvent A:B=95:5 (solvent A, water, 0.1% trifluoroacetic acid; solvent B, acetonitrile, 0.1% trifluoroacetic acid) and maintained for 1 min. We then increased concentration of solvent B to 50% over a gradient for 24 min. Concentration was returned to 95% solvent A and equilibrated for 2 min. Flow rate was 1 mL/min and metabolites were tracked at 270 nm. During the run, the peak corresponding to pN-Phe was collected (FIG. 11A) and then submitted to UPLC-MS as previously described. A standard (FIG. 11B) demonstrates the elution time of pN-Phe and the corresponding peak for pN-Phe (MW=210) is shown in the MS peak at (M+1)=211. The de novo synthesis (pACYC-AroG+pCola-papABC+pZE-ObaC 24 h purified peak) sample (FIG. 11C) demonstrates similar elution time and MS peak at (M+1)=211.

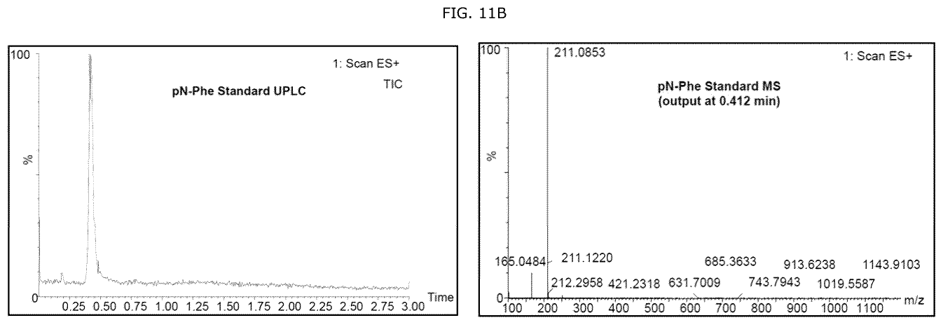

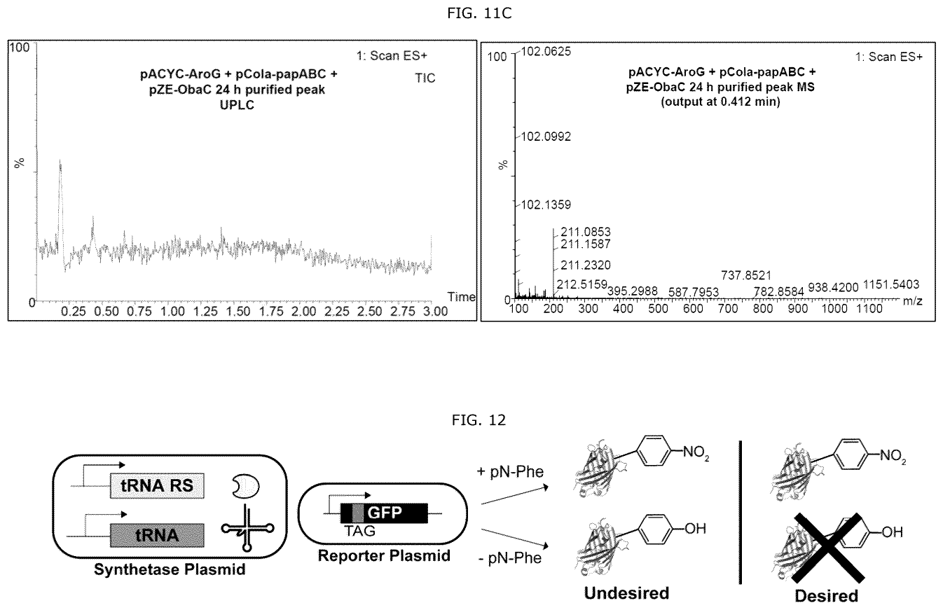

To investigate incorporation of pN-Phe, we used an NSAA incorporation assay commonly implemented in live cells (FIG. 12). Herein, a fluorescent reporter protein is chosen and its gene is modified to include an in-frame TAG sequence at the DNA level, resulting in an in-frame UAG codon at a designated location within the protein sequence. Upon co-expression with an engineered or natural aminoacyl-tRNA synthetase (AARS) and tRNA pair, where the tRNA contains an anticodon that pairs with UAG, the amount of fluorescent protein produced per cell can be indicative of the level of NSAA incorporation. Thus, measurement of fluorescence normalized by culture optical density (FL/OD) provides a high-throughput measurement of NSAA incorporation, as long as the FL/OD measurement remains low in the absence of NSAA. High FL/OD in the absence of NSAA indicates likely undesired background incorporation of a standard amino acid, whereas low FL/OD in the absence of NSAA and high FL/OD in the presence of NSAA indicates a desired result.

To perform an initial screen of AARSs for selective pN-Phe incorporation, we cloned MjTyrRS derivatives within pEVOL plasmids and co-transformed these alongside a pZE-GFP_1UAG plasmid harboring reporter protein into MG1655 (DE3). We cultured transformed strains in 300 μL LB broth in deep 96-well plates with 0.2% (wt/v) L-arabinose, 1 mM nsAA, 34 μg/mL chloramphenicol, and 50 μg/mL kanamycin. At mid-exponential growth (OD˜0.5), we added 0.2 nM ATC to induce transcription of RNA that requires UAG suppression to form full-length GFP. We grew these cultures for 18 h at 34° C. before pelleting them via centrifugation. To eliminate possible fluorescence or absorbance by free nsAAs in culture media, we washed cultures in PBS buffer before quantification of GFP fluorescence at excitation and emission wavelengths of 488 and 528 nm, respectively. For each synthetase, we performed this screen in the presence and absence of externally supplied nsAA given the tendency of several synthetases to accept natural aromatic amino acids (primarily L-Tyr) that are always present in cells, which results in varying degrees of background GFP expression. Our results (FIG. 13) demonstrate identification of multiple synthetases (NapARS, tetRS-C11, and pCNFRS) with desired activity and specificity towards pN-Phe rather than pA-Phe.

We then investigated the effect of pN-Phe concentration on the NSAA incorporation level for different synthetases. These experiments were performed just as described above, except with only 3 top-performing AARSs and with more supplemented pN-Phe concentrations tested. The results, repeated months apart (FIG. 14A in April 2020 and FIG. 14B in October 2020), demonstrate that while the incorporation level of pN-Phe is dose-dependent, even at doses as low as 0.1 mM pN-Phe the incorporation level is elevated above what is seen for 2 mM pA-Phe addition. Given that biosynthetic titers observed have reached ˜0.3 mM in the extracellular media, these experiments strongly suggest that the coupling of biosynthesis and incorporation of pN-Phe will be feasible to a skilled practitioner in the art.

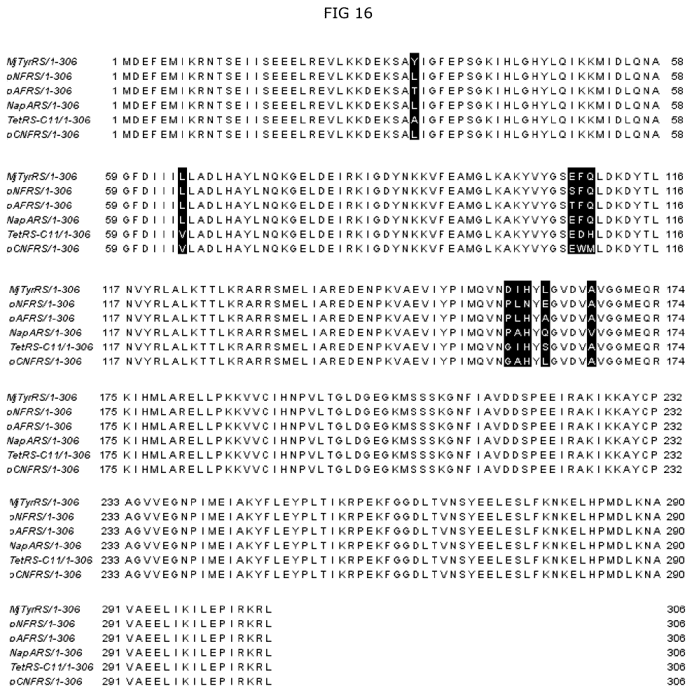

We then sought to pair the activity of the enzyme ObaC in culture with an AARS (tetRS-C11) and tRNA pair for pN-Phe incorporation starting from a pA-Phe precursor. To do so, we grew cultures we co-transformed E. coli MG1655 (DE3) with a plasmid containing a previously published engineered derivative of the Methanococcus jannaschii TyrRS (TetRS-C11)³, a pZE-ObaC construct expressing the N-oxygenase ObaC and a ubiquitin fused GFP reporter containing an amber suppression codon encoded on a vanillate inducible promoter system (pCDF-Ub-UAG-GFP). We cultured these strains at 37° C. in 50 ml of LB broth in 250 mL baffled shake flasks with 0.2% (wt/v) arabinose, 1 mM of either pN-Phe (FIGS. 15A-15B) or pA-Phe (FIG. 16), 25.5 μg/mL chloramphenicol, 37.5 μg/mL kanamycin, 71.3 uL streptomycin, and 0.2 nM ATC in a shaking incubator at 250 RPM. At an OD₆₀₀ of 0.5-0.8, we added 1 mM vanillate to induce transcription of mRNA that requires UAG suppression to form full-length GFP. We then grew cultures at 37° C. for an additional 18 h. We then purified the reporter protein using FPLC with a His-Trap column as previously described. We then concentrated the protein sample using a 10 kDa MWC Amincon column and diluted the sample 10:1 in 10 mM ammonium acetate buffer three times using the column. Then, we diluted the sample 10:1 in 2.5 mM ammonium acetate buffer concentrated the sample to about 200 μL. Protein in 2.5 mM ammonium acetate buffer was then submitted for intact protein MS using electrospray ionization (ESI-MS). For intact protein ESI-MS measurements, samples were submitted to a Waters Acquity UPLC H-Class coupled to a Xevo G2-XS Quadrupole Time-of-Flight (QToF) Mass Spectrometer. Protein sample was injected with an initial mobile phase of solvent A/B=85/15 (solvent A, water, 0.1% formic acid; solvent B, acetonitrile, 0.1% formic acid) held at 85/15 for 1 minute followed by a gradient elution from (A/B) 85/5 to 5/95 over 5 min. Flow rate was maintained at 0.5 mL min−1. Spectrum was analyzed from m/z 500 to 2000 and the spectra was deconvoluted using maximum entropy in MassLynx. The pN-Phe supplemented control sample (FIG. 15A) confirmed a mass of 37307 Da (theoretical MW) and the pA-Phe supplemented sample (FIG. 15B) confirmed mass of 37308 Da (theoretical MW).

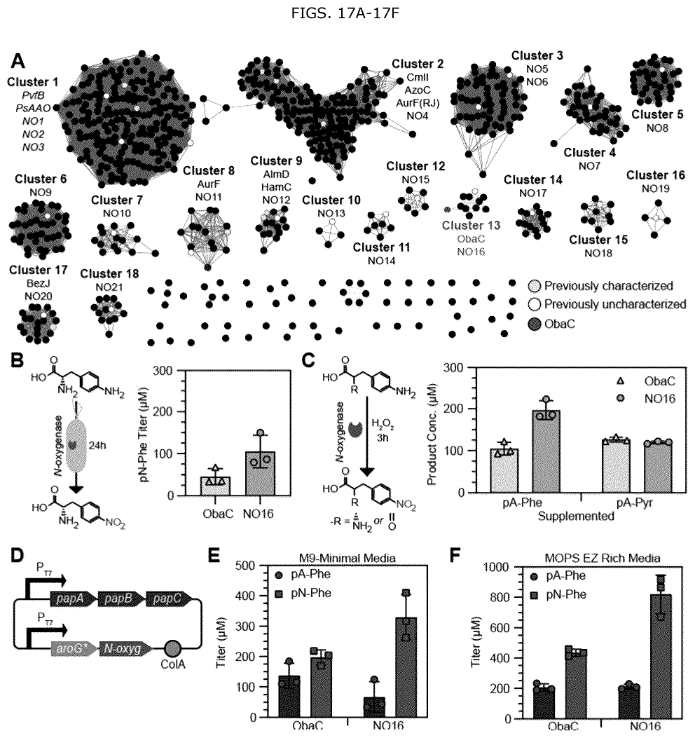

To further optimize the biosynthetic system, we hypothesized that we could increase pN-Phe titers by identifying a more active N-oxygenase to improve conversion of pA-Phe to pN-Phe. Previously characterized non-heme diiron monooxygenases have demonstrated activity on diverse aromatic amines and compatibility with expression in E. coli. Thus, while ObaC was the only reported diiron monooxygenase to exhibit full oxidation of pA-Phe to pN-Phe, we hypothesized that additional N-monooxygenases with activity on pA-Phe or pA-Pyr could exist. To assess the broader space of candidate diiron monooxygenase enzymes, we generated a protein sequence similarity network (SSN) with 2,134 putative N-oxygenase sequences represented as 775 nodes. We visualized the SSN using Cytoscape and implemented a minimum alignment score cutoff of 100 for node edges. We observed several clusters with characterized N-monooxygenases found in four of these clusters (FIG. 17A). To sample this diversity, we selected 21 distinct sequences, NO1-NO21 (Table 5), from 18 different clusters for heterologous expression. Upon cloning these with a C-terminal hexahistidine tag, all the N-oxygenases except four (NO6, NO13, NO18, and NO20) expressed in the soluble fraction. We then assessed whether the soluble N-oxygenases were active on pA-Phe by supplementation to transformed and metabolically active E. coli MG1655. Under these conditions, only one additional N-oxygenase resulted in pN-Phe production (FIG. 17B). This N-oxygenase, which we labelled NO16, was in the same cluster, cluster 13, as ObaC and was the only protein to reside in a predicted bacterial gene cluster similar to obafluorin. Structurally, ObaC (SEQ ID NO: 7) and NO16 (SEQ ID NO: 33) are quite similar, sharing 73% sequence identity.

We next sought to compare the performance of ObaC and NO16 on substrates of interest in vitro and in the full pathway context in vivo. We purified NO16 and found that, in comparison to ObaC, NO16 exhibits a two-fold increase in pN-Phe production when supplied with pA-Phe, converting 1 mM substrate to 196±23 μM over 3 h (FIG. 17C). Conversion of pA-Pyr to pN-Pyr was comparable for ObaC and NO16. We then compared ObaC or NO16 in the full pathway context using a single plasmid expression system (FIG. 17D), initially using M9 minimal media supplemented with 1.5% glucose. This plasmid expressed feedback resistant aroG, and the papABC genes. We observed an improved pN-Phe titer at 24 h when co-expressing NO16 (330±75 μM), as compared to 197 +25 μM using ObaC (FIG. 17E). This system differed from previous figures in that it used a chorismite overproducing background strain (NST37 ΔPheA). The lower final titer of pA-Phe observed using NO16 lends further support to the notion that increased N-oxygenase activity improved pathway selectivity. To explore the role of culture medium in influencing titer, we also tested a rich defined media (MOPS EZ Rich with 1.5% glucose). Using this media, we achieved an even greater pN-Phe titer of 820±130 μM achieved after 24 h using NO16 compared to a titer of 435±23 μM using ObaC (FIG. 17E).

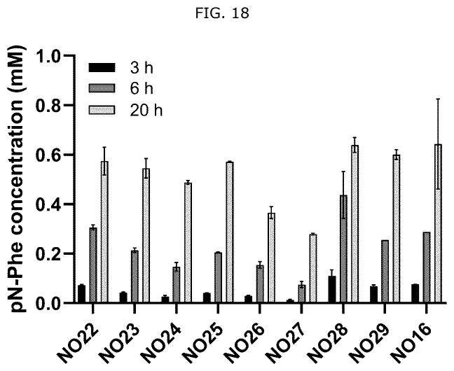

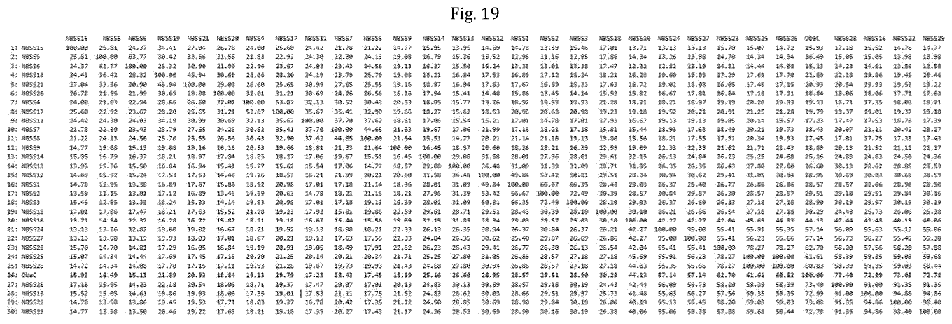

To assess whether additional N-monooxygenase enzymes could provide even greater improvement in pA-Phe conversion, we next focused on the remaining sequences from cluster 13 from the sequence similarity network which contained ObaC and NO16. To assess whether the pA-Phe oxidizing activity was shared by all enzymes of this cluster, we cloned and expressed these enzymes NO22-NO29 (SEQ ID NOS: 22-29) (Table 5) with a sequence similarity cutoff at 55% of the amino acid sequence of NO16 (SEQ ID NO: 33) in an E. coli MG1655 strain in a pZE vector under control of an anhydrotetracycline inducible promoter. We grew the strains to mid-exponential phase and added pA-Phe and induced enzyme expression. Under these conditions, all the tested N-oxygenases NO16 and NO22-NO29 exhibited conversion to pN-Phe within 6 h, with the dynamics of pN-Phe synthesis comparable across variants (FIG. 18). This data demonstrates that pA-Phe oxidizing activity is shared across enzymes found within cluster 13 at the selected sequence similarity cutoff. Here, these enzymes NO 16 and NO22-NO29 were found to have greater than 55% identity, whereas all other N-oxygenases tested outside the cluster had <45% identity (FIG. 19). Thus, we have established a clear set of natural N-oxygenases that exhibit pA-Phe oxidizing activity for our desired pathway.

In summary, by using the previously unidentified N-oxygenase NO16, we have achieved titers of >300 μM in M9-glucose media and >800 μM in a rich media.

### Conclusion

To summarize, we have demonstrated that pN-Phe is a fairly stable and non-toxic metabolite at relevant concentrations in E. coli. We have identified the amine mono-oxygenase ObaC is capable of catalyzing oxidation of pA-Phe and pA-Pyr in culture for toward the synthesis of pN-Phe and we have demonstrated de novo biosynthesis of pN-Phe from glucose carbon feedstock in a heterologous pathway expressing ObaC in addition to the papABC operon from S. venezuale in E. coli. We have additionally identified three AARSs that paired to tRNACUA can selectively incorporate pN-Phe. We have coupled one of these AARSs (tetRS-C11) with expression of its tRNA pair and ObaC and demonstrated pN-Phe synthesis and incorporation into proteins from pA-Phe precursor.

TABLE 1
Sequences of heterologous proteins involved in pA-Phe biosynthesis

| SEQ | | |
|---|---|---|
| ID | Protein | |
| NO | Names | Amino Acid Sequences |
| 1 | PapA (S. | MRTLLIDNYDSFTHNLFQYIGEATGQPPVVVPNDADWSRLPLEDFDAIV |
| | venezuelae) | VSPGPGSPDRERDFGISRRAITDSGLPVLGVCLGHQGIAQLFGGTVGLA |
| | | PEPMHGRVSEVRHTGEDVFRGLPSPFTAVRYHSLAATDLPDELEPLAWS |
| | | DDGVVMGLRHREKPLWGVQFHPESIGSDFGREIMANFRDLALAHHRAR |
| | | RDAADSPYELHVRRVDVLPDAEEVRRGCLPGEGATFWLDSSSVLEGAS |
| | | RFSFLGDDRGPLAEYLTYRVADGVVSVRGSDGTTTRTRRPFFSYLEEQLE |
| | | RRRVPVAPDLPFEFNLGYVGYLGYELKAETTGDPAHRSPHPDAAFLFADR |
| | | AIALDHQEGCCYLLALDRRGHDDGARAWLRETAETLTGLAVRVPAEPTP |
| | | AMVFGVPEAAAGFGPLARARHDKDAYLKRIDECLKEIRNGESYEICLTN |
| | | MVTAPTEATALPLYSALRAISPVPYGALLEFPELSVLSASPERFLTIGADG |
| | | GVESKPIKGTRPRGGTAEEDERLRADLAGREKDRAENLMIVDLVRNDLN |
| | | SVCAIGSVHVPRLFEVETYAPVHQLVSTIRGRLRPGTSTAACVRAAFPGG |
| | | SMTGAPKKRTMEIIDRLEEGPRGVYSGALGWFALSGAADLSIVIRTIVLA |
| | | DGRAEFGVGGAIVSLSDQEEEFIEIVVKARAMVTALDGSAVAGAR |
| 2 | PapB (S. | MTEQNELQRLRAELDALDGTLLDTVRRRIDLGVRIARYKSRHGVPMMQP |
| | venezuelae) | GRVSLVKDRAARYAADHGLDESFLVNLYDVIITEMCRVEDLVMSPSCTK |
| | | EVV |
| 3 | PapC (S. | MSGFPRSVVVGGSGAVGGMFAGLLREAGSRTLVVDLVPPPGRPDACLV |
| | venezuelae) | GDVTAPGPELAAALRDADLVLLAVHEDVALKAVAPVTRLMRPGALLADT |
| | | LSVRTGMAAELAAHAPGVQHVGLNPMFAPAAGMTGRPVAAVVTRDGPG |
| | | VTALLRLVEGGGGRPVRLTAEEHDRTTAATQALTHAVILSFGLALARLGV |
| | | DVRALAATAPPPHQVLLALLARVLGGSPEVYGDIQRSNPRAASARRALAE |
| | | ALRSFAALIGDDPDRAEDPDRADDPDRTDNPGHPGGCDGAGNLDGVFE |
| | | ELRRLMGPELAAGQDHCQELFRTLHRTDDEGEKDR |
| 4 | ObaD (P. | MFKTLIIDNFDSFTYNLYQYMGQVTGEEPDVFTNDASPHDIDLGRYDCII |
| | fluorescens) | VSPGPGTPKRRQDVGISEDMIRDAHVPLLGVCLGHQCMAHVHGMDVD |
| | | HAPEPMHGRVRHIRHNNEGVFKGLPVDMPVVRYHSLVVKALKGPFELSA |
| | | WDENGMIHGIRHTERPLYGIQFHPESICTDSGLDLLRNFRDIAHRHKLE |
| | | RLPR |
| 5 | ObaE (P. | MTTFDVEVRALDYNPDPLRVFRSEFLASPRHFFLESSVVKPGFSRFSFMG |
| | fluorescens) | DSHGRLAETITYDTSSRSVRVERSDGVTREPTSDFLELMAARLNHYHCE |
| | | QPQWLPFDFNLGYVGLLGYELKCETLGAQAYASHSHDAAFILATRMIAF |
| | | DHAEQRCYLLYLVEHDEDRQDAAQWFDQVQARLREQPQVAEPVSRQR |
| | | KLSLPQVEAWIQEHACIRHSKQRYIDKINEAQREIIDGETYEVCLTNLIEF |
| | | AFADSSFDLYCVMRELTPAPHAGYFSIPDFQIISSSPERFLKIDRHHQVE |
| | | AKPIKGTRPRGRCAEEDQELIEQMRGDEKDRAENLMIVDLLRNDLGQVC |
| | | TIGSVRVPALFAVETYSHVHQLVSTISGQLKPSLSAVDCVRATFPGGSMT |
| | | GAPKKRTMEIIDRLEEGPRGAYSGSLGWFGLGGACDLNIMIRSITVDAQ |
| | | VARFGVGGAITSLSDPLGEYIETMVKASGVVEAVTQLRSTSV |
| 6 | ObaF (P. | MSLSSPHRHAVVVGILGSIGQLLANQLSIAGYSVTGIDIAVDDQSAQPH |
| | fluorescens) | TVIQGDVLRPGNEIKQRLGDAQILVLALAQNVLSEALPQLLPSLRSDCLIV |
| | | DTLSIKSEFADFVATLDVAQPMVGINPMFSGDLDPAGRPVAVVTYRDGD |
| | | GDAVARLVEWLHSWPANVFQMTASEHDRTMAYLQTLGHALVMGMGLT |
| | | LAESAAPLENLFELAPPPFKVMLALLARMTKNHPDVYWEIQSNNPYSQEI |
| | | RSRMLAQLGKLDDRVNSGSRLDYHVSMAMLRNALKPLNPGLENTSRHL |
| | | FEQLDQAPKAIEGAPESLADYRQRIDHIDDQLVDLLGQRLSLIREVAQSK |
| | | KDHQTAVMQPNRVVQVVERCKARGRRHHIRESLIEQLYGLIIDEACQIE |
| | | YDVIGGPRESLYEASPSAFTSSAEKTQ |

TABLE 2
Sequences of heterologous proteins overproduced
and purified in this study

| SEQ | | |
|---|---|---|
| ID | Protein | |
| NO | Names | Amino Acid Sequences |
| 7 | ObaC (P. | MTMITPSLHACRSMPESQLLNKITDTWYAKATVRSTPRILVPDYSSEQL |
| | flourescens | IYPVARCSICEHPLVLELGPQVRSYILTQAAYQFLYGVGLLETKFVIQCCL |
| | with beta- | DMLHNNIKDISDAAKLQALTVIVDEGYHAHVALDYIIQMKKKSAIEPLE |
| | galactosidase | VPQTNRKLDATARAYASLPESMRMDFQLLAVTLAENVLTDEVANLGRE |
| | fusion | RELAQSFTTLMMDHVRDEGRHSRFFADLMKERWPQLPRATQEHFGLM |
| | underlined) | LPAYLDDFLGADLSRGFERKILAHCGLTEAQAEQVIHESDPHFSTDQAR |
| | | MKKSILQRIYRLLNQIGVLELDSVKDAFSDRNYVTTGGGSHHHHHH |
| 8 | Ub-UAG-GFP | MQIFVKTLTGKTITLEVESSDTIDNVKSKIQDKEGIPPDQQRLIFAGKQL |
| | (Synthetic, | EDGRTLSDYNIQKESTLHLVLRLRGG(*)LFVQELASKGEELFTGVVPIL |
| | NSAA | VELDGDVNGHKFSVRGEGEGDATNGKLTLKFICTTGKLPVPWPTLVTT |
| | incorporation | LTYGVQCFSRYPDHMKQHDFFKSAMPEGYVQERTISFKDDGTYKTRAE |
| | site shown | VKFEGDTLVNRIELKGIDFKEDGNILGHKLEYNFNSHNVYITADKQKNG |
| | with (*)) | IKANFKIRHNVEDGSVQLADHYQQNTPIGDGPVLLPDNHYLSTQSVLS |
| | | KDPNEKRDHMVLLEFVTAAGITHGMDELYKLEHHHHHH |

TABLE 3
Sequences of native E. coli transaminases anticipated to
contribute to the pathway

| SEQ | | |
|---|---|---|
| ID | Protein | |
| NO | Names | Amino Acid Sequences |
| 9 | TyrB (E. | MFQKVDAYAGDPILTLMERFKEDPRSDKVNLSIGLYYNEDGIIPQLQAVAEAE |
| | coli) | ARLNAQPHGASLYLPMEGLNCYRHAIAPLLFGADHPVLKQQRVATIQTLGGS |
| | | GALKVGADFLKRYFPESGVWVSDPTWENHVAIFAGAGFEVSTYPWYDEATN |
| | | GVRFNDLLATLKTLPARSIVLLHPCCHNPTGADLTNDQWDAVIEILKARELIPF |
| | | LDIAYQGFGAGMEEDAYAIRAIASAGLPALVSNSFSKIFSLYGERVGGLSVMC |
| | | EDAEAAGRVLGQLKATVRRNYSSPPNFGAQVVAAVLNDEALKASWLAEVEE |
| | | MRTRILAMRQELVKVLSTEMPERNFDYLLNQRGMFSYTGLSAAQVDRLREEF |
| | | GVYLIASGRMCVAGLNTANVQRVAKAFAAVM |
| 10 | AspC | MFENITAAPADPILGLADLFRADERPGKINLGIGVYKDETGKTPVLTSVKKAEQ |
| | (E. coli) | YLLENETTKNYLGIDGIPEFGRCTQELLFGKGSALINDKRARTAQTPGGTGAL |
| | | RVAADFLAKNTSVKRVWVSNPSWPNHKSVFNSAGLEVREYAYYDAENHTLD |
| | | FDALINSLNEAQAGDVVLFHGCCHNPTGIDPTLEQWQTLAQLSVEKGWLPLF |
| | | DFAYQGFARGLEEDAEGLRAFAAMHKELIVASSYSKNFGLYNERVGACTLVA |
| | | ADSETVDRAFSQMKAAIRANYSNPPAHGASVVATILSNDALRAIWEQELTDM |
| | | RQRIQRMRQLFVNTLQEKGANRDFSFIIKQNGMFSFSGLTKEQVLRLREEFG |
| | | VYAVASGRVNVAGMTPDNMAPLCEAIVAVL |
| 11 | IlvE (E. | MTTKKADYIWFNGEMVRWEDAKVHVMSHALHYGTSVFEGIRCYDSHKGPVV |
| | coli) | FRHREHMQRLHDSAKIYRFPVSQSIDELMEACRDVIRKNNLTSAYIRPLIFVG |
| | | DVGMGVNPPAGYSTDVIIAAFPWGAYLGAEALEQGIDAMVSSWNRAAPNTIP |
| | | TAAKAGGNYLSSLLVGSEARRHGYQEGIALDVNGYISEGAGENLFEVKDGVL |
| | | FTPPFTSSALPGITRDAIIKLAKELGIEVREQVLSRESLYLADEVFMSGTAAEIT |
| | | PVRSVDGIQVGEGRCGPVTKRIQQAFFGLFTGETEDKWGWLDQVNQ |

TABLE 4
Sequences of Methanacoccus jannaschii tyrosyl tRNA synthetase
derivatives

| SEQ | | |
|---|---|---|
| ID | Protein | |
| NO | Names | Amino Acid Sequences |
| 12 | MjTyrRS | MDEFEMIKRNTSEIISEEELREVLKKDEKSAYIGFEPSGKIHLGHYLQIKK |
| | (M. | MIDLQNAGFDIIILLADLHAYLNQKGELDEIRKIGDYNKKVFEAMGLKAKY |
| | jannaschii) | VYGSEFQLDKDYTLNVYRLALKTTLKRARRSMELIAREDENPKVAEVIYPI |
| | | MQVNDIHYLGVDVAVGGMEQRKIHMLARELLPKKVVCIHNPVLTGLDGE |
| | | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |
| 13 | pNFRS (M. | MDEFEMIKRNTSEIISEEELREVLKKDEKSALIGFEPSGKIHLGHYLQIKK |
| | jannaschii, | MIDLQNAGFDIIILLADLHAYLNQKGELDEIRKIGDYNKKVFEAMGLKAKY |
| | mutations | VYGSSFQLDKDYTLNVYRLALKTTLKRARRSMELIAREDENPKVAEVIYPI |
| | underlined) | MQVNPLNYEGVDVAVGGMEQRKIHMLARELLPKKVVCIHNPVLTGLDGE |
| | | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |
| 14 | pAFRS (M. | MDEFEMIKRNTSEIISEEELREVLKKDEKSATIGFEPSGKIHLGHYLQIKK |
| | jannaschii, | MIDLQNAGFDIIILLADLHAYLNQKGELDEIRKIGDYNKKVFEAMGLKAKY |
| | mutations | VYGSTFQLDKDYTLNVYRLALKTTLKRARRSMELIAREDENPKVAEVIYPI |
| | underlined) | MQVNPLHYAGVDVAVGGMEQRKIHMLARELLPKKVVCIHNPVLTGLDGE |
| | | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |
| 15 | NapARS (M. | MDEFEMIKRNTSEIISEEELREVLKKDEKSALIGFEPSGKIHLGHYLQIKK |
| | jannaschii, | MIDLQNAGFDIIILLADLHAYLNQKGELDEIRKIGDYNKKVFEAMGLKAKY |
| | mutations | VYGSEFQLDKDYTLNVYRLALKTTLKRARRSMELIAREDENPKVAEVIYPI |
| | underlined) | MQVNPAHYQGVDVVVGGMEQRKIHMLARELLPKKVVCIHNPVLTGLDGE |
| | | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |
| 16 | TetRS-C11 | MDEFEMIKRNTSEIISEEELREVLKKDEKSAAIGFEPSGKIHLGHYLQIKK |
| | (M. | MIDLQNAGFDIIIVLADLHAYLNQKGELDEIRKIGDYNKKVFEAMGLKAKY |
| | jannaschii, | VYGSEDHLDKDYTLNVYRLALKTTLKRARRSMELIAREDENPKVAEVIYPI |
| | mutations | MQVNGIHYSGVDVAVGGMEQRKIHMLARELLPKKVVCIHNPVLTGLDGE |
| | underlined) | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |
| 17 | pCNFRS (M. | MDEFEMIKRNTSEIISEEELREVLKKDEKSALIGFEPSGKIHLGHYLQIKK |
| | jannaschii, | MIDLQNAGFDIIIVLADLHAYLNQKGELDEIRKIGDYNKKVFEAMGLKAKY |
| | mutations | VYGSEWMLDKDYTLNVYRLALKTTLKRARRSMELIAREDENPKVAEVIYPI |
| | underlined) | MQVNGAHYLGVDVAVGGMEQRKIHMLARELLPKKVVCIHNPVLTGLDGE |
| | | GKMSSSKGNFIAVDDSPEEIRAKIKKAYCPAGVVEGNPIMEIAKYFLEYPL |
| | | TIKRPEKFGGDLTVNSYEELESLFKNKELHPMDLKNAVAEELIKILEPIRKR |
| | | L |

TABLE 5
Sequences of N-oxygenase derivatives

| SEQ ID | Protein | |
|---|---|---|
| NO | Names | Amino Acid Sequences |
| 18 | NO1 | MNASDYRSIAEDWEHRATIRTRPRRVLENDDNLIYPLCRQPLVACATFLEHC |
| | | SQWRDFVLVQSFYKFINDVVIFETEIVDKTARSIAKNRFAVPFPLACRYDAMT |
| | | IVVDEDYHALVALDFLQQTVEMTGIQPLELPKQIELGRALPAAQAQAPAHLH |
| | | DAVELIAVAIAENTVTHDVAAFAKDDSVKSSVRGMMADHLSDEGRHAQFW |
| | | TRLVRLYWQVASPQDRDSIALVLPTFLAQYLTNDLQASFDLQLIEHLDVSPGI |
| | | RHALREEINALDFPITRQHPLIGNITRFLHHSGLLQTPSVAQALDDYLPPAGS |
| | | AA |
| 19 | NO2 | MNAVDYCSFADAWEERATIRTRPRRQVEDDQRLIYPLSRQPLVLSATFVREC |
| | | PQQRDFALVQSLYKFIHDVVIFETEIVNHTASAIARGRFAHDFPFACRYDAMT |
| | | VVVDEDYHALVAMDFMQQTIELTGITPVALPSEIELSRAIPAALAQVPERLRQ |
| | | AAELICVAIAENTLTNDVAAFARDTSVKESVKGLMADHLQDEGRHATFWQR |
| | | LVRIYWQAAPADDQASLAHMLPTFIERYLTNDIQKAFDLALIEHLHPDPAVRQ |
| | | ALREEAQGLAFPISRQHPLLGNILRFFKGSSMLDSPCVRDALVAYLP |
| 20 | NO3 | MKAEDYVSFVDAWEARATIRTRPRRIVEDDERLIYPLSRQPLVLSETFTRQCP |
| | | ALRDYALVQSLYKFINDVVIFETEIVDKTARSIAKDRFAIRFPFACRFDAMSVV |
| | | VDEDYHALVAMDFMQQTIALTGIEPITLPTQIELSRAIPAALAQAPEHLRSAVE |
| | | LICVAIAENTVTHDVAAFAKDDTVKQSVKGLMADHLLDEGRHSGFWTRLVR |
| | | IYWQTAAPADRAAIAQILPVFIDQYLTNEIQNGFDFILINHLPVAEVVKQALKA |
| | | ETLALSFPISSHHPLVGNIVRFFRNSTMLEAPCVRQALVDYLPAGEVYA |
| 21 | NO4 | MTDHQAANRRAARLRPPELPEHDAADPAAAAVIRGLTRSWPRRAAVKKTEP |
| | | DRDALFEPARDDFPDTLLPFHTHPRYAALDEPARARLRAWGWIAYNKNVIDI |
| | | EQYVVNPGFGLITSDVFGLAPPDTVVLGVMQAMVDEQYHTLMHQNANALTR |
| | | RRRGLTLPDAVLPACRTLRAHRAAVQRATDPRHEALVQLAFTTVAETSISAYL |
| | | ALFIGDETVQPVNRATVSVHRRDELCHATLTGELFTTVFTELDAADRERLFEA |
| | | LAAGIDAFTGGDMTTWAAIVAHEGIPGGREMIADTADATAHQPVVQDCSAI |
| | | HRLCASVGAGEDFDAHRARRLQT |
| 22 | NO5 | MLEKLSVLWSTRAAVNNKQPTYSELAFDPGKNDFCESLLPFRNHPAWIEASP |
| | | EMKSTCLSYAWIIYNLKTIYIECDIVTPACEDIIKTPPSATRNRALLQESISEAL |
| | | LDEALHTKMSVTACNYIYHHRDLAHLNFTDFNLIKWKNWVLSSCSAEWERR |
| | | LTRFGIACASETLITDYLKTLAEDSSVQSICHQVTKTHAMDEWSHSSVFSYV |
| | | AADLISTLSQHEKTHLKAIIKKTVSMFANNEMGAWEMAFSHARLPRYADIIH |
| | | DTATLGEVNVYTESVDKLLSRIGLEETH |
| 23 | NO6 | MDRLNCGTDDVVKNMLAKLSGLWKTRAAVNNPQPDYWGLAFDPNKQDFS |
| | | LSLLPFRHHQAWLEAPQDLQSKCLSYAWGIYNLKTIYIECDVVVPACEDIIKT |
| | | PPPSQNRALLQDVMSQALLDEALHTRMSIMACNYIYDMRKLTPLDFSAFNLV |
| | | TWREALLANCTAEWERRLTRFGVACASETLITDYLKTMAEDTSIQAVCHEVT |
| | | RTHAIDEWSHSSVFSFVASDIVHGLSRTEREYLRAIILRTVQMFANNEMGA |
| | | WARVFSMLQMPHASEILHEVGDNNEAPNTPSKP |
| 24 | NO7 | MTITSDAPGPAGSGPDTLRRLVAAWPRRATIRTDLDKVTDPGPYDPALMDYP |
| | | VSLLPFAGHPSYLAASDEQRLQVNTLAWLAYNERVIAAEEHVANPTFEKLSH |
| | | GVFPGADGVDAREAIQQSHIDEVWHTYMHMLAMQRSREARRVTAEPNDYL |
| | | HPVTNRRLYRLADEASEQWERDLLYLLWTVVGEVSINAFLDLVAGDRTIQPG |
| | | HALIARLHARDEAAHGPILAEVMKDVFVHLNAAQREVLVRRLPEAVTAFGAE |
| | | DYELWLRILDFAGFADARAIVEDSRALPGAELMLTDFAPIQRLIRELGVDDRV |
| | | DLDLPAPS |
| 25 | NO8 | MRYEESGVPGGLTTWAARGWAEEHGIGSATLRRLVRAWPRRAAVTTRPEV |
| | | MEESAAYDPALPDYPLEIVPFAEHPRFLAARPDQRRQVLTGLWIGYNERVIAT |
| | | EHLIAEPAFDLVMCGVFPGGDDPLIRQSVQQAVVDESFHTYMHMLAIARTRE |
| | | LRDVRTRPEQPPLVTYRRLCALLATMPEEWERHVAVLVWGAVAETCINSLLA |
| | | LLARDESIQPAHSLITTLHLRDETAHGSVVTEVVRELYARMNAAQRRTLVRCL |
| | | PLALEAFAEQDLSTLRLELATAGVHGADEIVGDLRATSTGRRLVRDFSGAQR |
| | | MVEQLGLAGEVDFDFPERPAWSPAAATSH |
| 26 | NO9 | MSALSREPVFQFSLNDWNTRASVRTSTHDYRLPANVQEQLETRHWFPPTFL |
| | | PYLAHPVIEAAGRSMLHRLTARHLVHFLDYTTLLEHRIVNRAVETIVHGELPV |
| | | AVPAPMKTAALQLYTDEGYHALFSNQVAEQIASLYGITKRPVMPRRITRMNLL |
| | | IARTDQQQRPLACFLLGFVSETIIARELLDVCRDSLVSGVNDMLRDHLTDEA |
| | | RHSRYFTEVFHYLWLHLDPRQRTFTATTLLDILEIFFEVDEHWLQESLCGADI |
| | | AGTTVMEILGTITTAQASRQRARTGSLATMSALKKAGFFAEPQNQTLFARAG |
| | | LIDG |
| 27 | NO10 | MISTNSQAAAAEHDNVSRWYANATVRHRPRIMVPDSSKGEFIFPRSRQLLS |
| | | EHPLVTARGHDAVAYVLAQSAYKYMYEIGLLETRFVIDCSLNIVNNQIKGFND |
| | | DEKREALTIVIDEGYHAYVALDFIIQMKSLTGIQPIEVPETNGNLQAVSQAMD |
| | | ALPAFMHNDFQLISVCLAEHTLTKDLLSIGREKDATQAFTQVMTDHVSDEGR |
| | | HANYFARLMKQHWVSLPDATKTTIGFMLPNYLDEYLAGDGERSFDKKVLLAL |
| | | GMSEAEAMRVIDDTHPAFMDNLHSYVSVTKANLVKLLARCGVLDHAPTRAS |
| | | FVAHGVEL |
| 28 | NO11 | MQTAIEIAKVDHQLIVRLNQAWTRRATVCSPDKWSVDEVFDPARPDYPEGM |
| | | VPFFHHPKFQALSEALKSRVVTWGWIGYNLRTVTAEEHVVNPALSVIANQYL |
| | | GKDDWHFREAMQQTLIDEHYHTLMHLRAIERTRLERALDQDLDLPPSVTYLR |
| | | LLALRETLTEQWQKDLAAITFAVVAEISVNAYLDLLADDQTIQPQNRRVAEQ |
| | | HNRDEYAHSKVLAEVAKVMYGNMQPPQRAFFARTLSQALSAFIAQDYSMW |
| | | EAILTQLGVEDAAQIIADTRESNKNATIMRDYSGLHRLAQDLGISEQIDFDFR |
| | | PTLKTTAAA |
| 29 | NO12 | MMTLQSYKSHAESWEQRATIRSRPRRIVEDDGLHYYPLERQPLCAHPAIVDA |
| | | GDAVRDFILLQSFYKYINDVIIFETEIVNATALKIARGRFAHDFPFACRHDAM |
| | | SVVIDEDYHAYVAMDYLRQVEVHTGVAALAANHEIELSRAIPRALERVAPPYR |
| | | DGMELLAVAISENTVTAEVAAFSRDTTLKRSVKGVMSDHLADEGRHSVFWI |
| | | NLVKLYWSEIDEAARSALGETLPVFLKEYLSAELQLAFDRRLIDALDLPAATR |
| | | REIAGDMVGAYPITSQHPMIVNIRNFFKLSGLLDHAPTRAALASVL |
| 30 | NO13 | MSPRDYKSKELVWDSLAATRSKPRRVLPEGDVVGHLYPPAKAALLTHPLMK |
| | | NLPPETLRLFFIHSAYKFMGDIAIFETETVNEVAMKIANGHTPITFPDDIRHDA |
| | | LTVIIDEAYHAYVARDFMRQIEQRTGVKPLPLGTETDLSRAMAFGKHRLPETL |
| | | HGLWEIIAVCIGENTLTKDLLNLTGEKSFNEVLHQVMEDHVRDEGRHAVLFM |
| | | NVLKLVWSEMEESARLAIGQLLPEFIREYLSPKMMAEYERVVLEQLGLAAEHI |
| | | ERILSETYSEPTLEDFRARYPLSGYLVYVLMQCDVLSHAPTREAFRRFKLLAH |
| 31 | NO14 | MARMMNNAKSKIGEQILTLSKDGLTTGFTRQLQIEWEKRATVRSRPKVVYD |
| | | KEAKGYYYPISRTPLAIHPTVMAKGERAIEFLLIQSLYKYSNDIATIETRVVNET |
| | | IIKVLTNQMPIEFNKEQKINLYTIMVDEAYHAYVAFDAMLQIEDHTGVEALPL |
| | | PEVIEIEKAITKVKEKLDNKYHNIFDLICVCLAENTLTKEIITMTDQEETHPFFQ |
| | | KIIKDHFADEKRHSGIFYKLLEFIWINLNEEYKEHIGEIIVDFIEEYLGLTIQINF |
| | | DKQVLSKIGFSDKEASKIVEDTYGGFKLTKDHPMLKNIIMLLDKAGVFDQFT |
| | | LPKFEEKEWA |
| 32 | NO15 | MFQHSTQDDLYRSNDATDERLIKIERSWHKRAQIKLDSQRPDIQLDLQAPD |
| | | FLEALLPFAHDPHYQNYPHALKLLILSAGWVIYNQKTIAIETEIICPSCIDLLQL |
| | | GSRLSSASAATISETLADEAYHTLFSINMCELAITQRGMRIKWPELELTRHLS |
| | | QAQATLSEADFKIYRLAFSLVSETFISDYLADLSDAPEIQPVFRHVVALHKKD |
| | | ELVHKHIFPLFVQQVVGGFNAQQTALFTRGIANSIRIFPMREISAWRIILPQLL |
| | | EVFGDQSLLLPTQFTETGEADYSAMPEILSSIGIHPEAVLPEPQMMSEPAITV |
| 33 | NO16 | MSLDQINDHWYAKATVRSTPRIIVPDYDGEQLIYPVSRCAMCEHPIIQAHGK |
| | | RIKNYVLTQAAYQFLYNVGLLETKFIIQCCLDMLHDKFPGIGEVEKLQALTVII |
| | | DEGYHAHVALDYLTQMNEKSGIEAIEVPQTNQKLDATARAYAQLPESLRTEF |
| | | QLLAVTIAENVLTDEVANLGRERELSQSFTTLMVDHVRDEGRHSRFFADVMK |
| | | KRWPTLPLSTRERFGMMLPAYLDDFLNVDPERRFERRMLAHCGLSPEQAEQ |
| | | VIHESDPTFHADHARMKKSILQRLYRLLRQMGILDLPQVREAFADRDYVSA |
| 34 | NO17 | MTGHLPEHDASDPVESAVIDGLVRSWPRRATVRRTEPELDIPTMDDLYDDT |
| | | REDYPEVLLPFAGHPSWERLDEDVRTRVRAWAWIAYNKNVVDIEQDVVTPG |
| | | FGLLFRDVFGTGFSDADRAAVVQSMVDEEYHTLMHLNASALTRRRRGWALP |
| | | DTTLPESSTVRRHRQARAQAESPRAAALTTLAFATVAETSIADYLSLIAGDQT |
| | | IQPVHQATIALHRRDERAHASVATEMIAMVHDKLDSDDRSFLARALRDGVD |
| | | AFTATDGAVWSAILAAEQVPDADQMLRDVAEDPDRRHFLQDCSAINRLLAR |
| | | FGELEHR |
| 35 | NO18 | MSLIETTLKKWDRAAIVRSRPRRLLNEQDSQSTLFFPLERLPLCLHPRVAALG |
| | | EQALRYIQVQTLYHYLNGIANIELDIINDSSYTLYKNAVGVDFPLEMRLDALTV |
| | | VVDESYHALVALDLINQVEQMTSIAPLPMPKDTEASYALAQSLAMGDERLRE |
| | | LLRLVCVSLSEQALTTDLIDVINNEDIFPTFYLVMKDHVADEGRHARFSQQVL |
| | | SHIWRHSDAATKQALQQPILAFLEGYATDAIAKDFSACMLRQLGLTEAEIIDI |
| | | IADTYPATHVKPLETNNIVTEQMLVALNKSGILADLPIADALHATI |
| 36 | NO19 | MTTMSAPAIATGGTDAAHQHEQSEVKALLQRIGDGWIKRAKVRRDAVDLAF |
| | | DPARPDFLEELLPFHAHPLYQRLAPELKSKILSAGWIIYNEKTVAIESAIVSPS |
| | | CYDALDGRIPGLTDETSRQIVCETLVDEAYHLLLVANANRLTRARRGLEDLKI |
| | | PSFNLVTMMNREKSLQSEDWQKILVHLATSVVSEIFVSDYLHQLSDCAEIQP |
| | | MNMATVAAHRHDELAHSKIFTLMTKTFYPALNPREQAFFASVLPKPIEWFAD |
| | | LELDIWSSVLEQLRVPGAATIIADCRPLNAAARERLDYTGIVGLSHEVGILST |
| | | DAGRNSFAAQGIAI |
| 37 | NO20 | MTAAREVPEYERDLLVOLTLRWGKRVAVKKDELDLDGHFDAALPDFPEHLVP |
| | | VLGLPGADRLDRDARERILSAAWIAYNAKTAAIENEIILPACRLMLQERIPVR |
| | | RDDVAVDALHQTIIDEHYHILMCHNAVGVTRRRRGLEDVRFAPGVWSVVQ |
| | | GLNAVRAGLSGFDRDIVDIAFSLAAETTISGFLSALATAQEIQPMNRITTDLH |
| | | RRDESGHAVVFRELCGSLYRNLDGAQQRTFREALVTGLTAFRSADLEPWVT |
| | | VAAQGGFEIDTGQLSEWAASRPVPARDTGPLQLLLDDLGISHDLVEMVQR |
| 38 | NO21 | MTLTASIPGTQKTSQSRKLLSKMASNWSRRAKVVQAKKQNFENNSELVFDS |
| | | ARDEFRIDLLPFKDHPAFLEAPPEMQKQILSCGWIAYNEKTVDIESKVISPAC |
| | | SHIIYQEVPGSDDGIGREIASDTLVDEAYHILLVMQACRITREKRGLESLKLP |
| | | SFNLVKQMTLEKAKYSERWQKILVQIVTATVSEVFISDYLNLLAKDTNIQPM |
| | | NRLIVDTHRQDEMAHHSIFINLAKCIYSELSPQQQEFFADILPKAVRWFANS |
| | | ELEVWQTMLEYLGFSKTREVIADCAVTDEVNLSRIDYSAVVLLAKELGVLDS |
| | | QKGIDSFARQGLLK |
| 39 | NO22 | MSLDLINDHWYAKATVRSTPRIIVPEYDGEQLIYPVSRCAMCEHPIIQTHGK |
| | | HIRNYLLTQAAYQFLYNVGLLETKFIIQCCLDMLHDKFPGIGNEEKLQALTVII |
| | | DEGYHAHVALDYLTQMNEKSGIEAIEVPQSNQKLDATARAYAQLPESLRTEF |
| | | QLLAVTIAENVLTDEVANLGRERQLSQSFTTLMVDHVRDEGRHSRFFADLM |
| | | KKRWPTLPESTRERFGLMLPAYLDDFLDIDPERRFERRMLAHCGLTPEQAEQ |
| | | VIHESDPTFHADHARMKKSILQRLYRLLRQMGILDLPQVREAFADRDYVSA |
| 40 | NO23 | MLIAEQNEITAQWYQRATVRYTPRIIVPDYSDEELIYPLARCAVCEHPLVLER |
| | | GQATRAFILAQAAYQFLYGVGLLETKFVIQCSLDLLHNQIPGIGEFDKLQALT |
| | | VIIDEGYHAHVALDYIMQMQHQSGITPLQVPESNHKLDAAARASATLPPAMR |
| | | PDFQLLAVTLAENVLTEEIATLGRDKGLAQTFTTLMMDHTRDEGRHSGYFAD |
| | | LMKKRWTQLPQATRDQFTQMMPDYLEDFLGTDTTRQFERRILRACKFTPQQ |
| | | IEQIIAESESEFIDLHRQLTQKTRIRLDRLLKQIGVREPDTNTTATAHASDAA |
| 41 | NO24 | MNQAQENHPAPDGAISAWYEKATVRYTPRIIVPDYTTDALIYPTALSIISEHPL |
| | | VVARGKEIRSFILTQSAYRFLYGVGLLETKFVIQCSLDLVHDRIFPVDDMQRL |
| | | QALTVIIDEGYHAHVALDYIVQMREKSGIVPLSVPQTNRKLDATARVYATLPP |
| | | DLRMDFQLLAVTLAENVLTNEIATMGREKELTKSYITLMTDHVMDEGRHSNY |
| | | FNDLMRKRWAQLDATVQSTFGTLLPAFLDDFLGIDLHRAFEREMLDACGFQ |
| | | SSDVQRILEDTQETYSNNHATLSTRTKARLFRLAKTFGILDRDDVRDAFAAR |
| | | GYAFQ |
| 42 | NO25 | MLIAEQNDLTNHWYQRATVRHTPRIIVPDYSDEELIYPLARCAVCDHPMILAR |
| | | GQATRAFILAQAAYQFLYGVGLLETKFVIQCSLALLHHEIPGIDEFARLQALTV |
| | | VIDEGYHAHVALDYIIQMQARSGIVPLQVPQSNHKLDAAARASATLPEAMRA |
| | | DFQLLAVTLAENVLTDEIANLGRDKGLAQTFTTLMMDHVRDEGRHSNYFAD |
| | | LMKARWAHMPQATRDRFTAMLPDYLDDFLGTDPSRSFERKILRACEFSPAET |
| | | ERIIDETQGEFQALHDQLTAKTKIRLDRLLRQIGVPDPANADSAAALDAAV |
| 43 | NO26 | MGCDAPGQEKPMLIAEQNDLTNHWYQRATVRHTPRIIVPDYSDEELIYPLAR |
| | | CAVCDHPMILARGQATRAFILAQAAYQFLYGVGLLETKFVIQCSLALLHHEIP |
| | | GIDEFARLQALTVVIDEGYHAHVALDYIIQMQARSGIVPLQVPQSNHKLDAA |
| | | ARASATLPEAMRADFQLLAVTLAENVLTDEIANLGRDKGLAQTFTTLMMDHV |
| | | RDEGRHSNYFADLMKARWAHMPQATRDRFTAMLPDYLDDFLGTDPSRSFE |
| | | RKILRACEFSPAETERIIDETQGEFQALHDQLTAKTKIRLDRLLRQIGVPDPAN |
| | | ADSAAALDAAV |
| 44 | NO27 | MNQAEENHPAPDGAISAWYEKATVRYTPRIIVPDYTTDALIYPTALSIICEHPL |
| | | VVARGKEIRSFILTQSAYRFLYGVGLLETKFVIQCSLDLVHDRIFPVDDVERL |
| | | QALTVIIDEGYHAHVALDYIVQMRAKSGIAPLSVPQTNRKLDATARVYATLPP |
| | | ELRMDFQLLAVTLAENVLTNEIATMGREKELTKSYITLMTDHVMDEGRHSNY |
| | | FNDLMRKRWAQLDATVQSTFGTLLPAFLDDFLGIDPHRAFEREMLGACGFPS |
| | | GDVQRILDDTQETYSGNHAAVSTRTKARLFRLAKTFGILNRDDVRDAFAAR |
| | | GYAFQ |
| 45 | NO28 | MSLDLINDHWYAKATVRSTPRIIVPDYDGQQIIYPVSRCAMCEHPLIREHGP |
| | | ELKAYILTQAAYQFLYNVGLLETKFIIQCCLDMLHDKFPGIGDTEKLQALTVII |
| | | DEGYHAHVALDYLTQMNAKSGIEAIEVPQTNQKLDATARAYAQLPESLHKEF |
| | | QLLAVTIAENVLTDEVANLGRERELSKSFTTLMTDHVRDEGRHSRFFADLMK |
| | | KRWPTLPESTRERFGLMLPAYLDDFLDIDRERRFERRILAHCGLSPEQAEQVI |
| | | HESDPTFHADHARMKKSILQRLYRLLROMGILDLPQVREAFANRDYACA |
| 46 | NO29 | MCPPHLTGKKMSLDLINDHWYAKATVRSTPRIIVPEYDGEQLIYPVSRCAMC |
| | | EHPIIQAHGKHIRNYLLTQAAYQFLYNVGLLETKFIIQCCLDMLHDKFPGIGN |
| | | TEKLQALTVIIDEGYHAHVALDYLTQMNEKSGIEAIEVPQSNQKLDATARAYA |
| | | QLPESLRTEFQLLAVTIAENVLTDEVANLGRERELSQSFTTLMVDHVRDEGR |
| | | HSRFFADLMKKRWPTLPESTRERFGLMLPAYLDDFLDIDPERRFERSMLAHC |
| | | GLTPAQAEQVIHESDPTFHADHARMKKSILQRLYRLLRQMGILDLPQVREAF |
| | | ADRDYVSA |

The above disclosure generally describes the present invention. All references disclosed herein are expressly incorporated by reference. A more complete understanding can be obtained by reference to the following specific examples which are provided herein for purposes of illustration only, and are not intended to limit the scope of the invention. 5

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A recombinant cell for producing para-nitro-L-phenylalanine (pN-Phe), comprising one or more heterologous genes encoding one or more heterologous enzymes, and expressing the one or more heterologous enzymes and a native metabolite selected from the group consisting of chorismate, para-amino-phenylpyruvate (pA-Pyr) and para-nitro-phenylpyruvate (pN-Pyr), wherein the native metabolite is converted to the pN-Phe in the recombinant cell.

2. The recombinant cell of claim 1, wherein the native metabolite is the chorismate, wherein the one or more heterologous enzymes comprise PapA, PapB and PapC, and wherein the chorismate is converted to para-amino-phenylpyruvate (pA-Pyr) in the recombinant cell.

3. The recombinant cell of claim 2, further expressing an N-monooxygenase, wherein the pA-Pyr is converted to para-nitro-phenylpyruvate (pN-Pyr) in the recombinant cell.

4. The recombinant cell of claim 3, further expressing an aminotransferase, wherein the pN-Pyr is converted to the pN-Phe.

5. The recombinant cell of claim 2, further expressing an aminotransferase, wherein the pA-Pyr is converted to para-amino-L-phenylalanine (pA-Phe).

6. The recombinant cell of claim 5, further expressing an N-monooxygenase, wherein the pA-Phe is converted to pN-Phe.

7. The recombinant cell of claim 2, wherein the recombinant cell is E. coli.

8. The recombinant cell of claim 1, further comprising a target polypeptide and expressing a heterologous aminoacyl-tRNA synthetase and a transfer RNA, wherein the pN-Phe is incorporated into the target polypeptide in the recombinant cell without requiring exposure of the recombinant cell to exogenous pN-Phe.

9. A cell culture comprising the recombinant cell of claim 1 in a culture medium.

10. The cell culture of claim 9, wherein the culture medium has glucose as the sole carbon source for the recombinant cell.

11. The cell culture of claim 9, wherein the culture medium is not supplemented with exogenous pN-Phe.

12. A method of producing para-nitro-L-phenylalanine (pN-Phe) by a recombinant cell, wherein the recombinant cell comprises one or more heterologous genes encoding one or more heterologous enzymes, the method comprising
(a) expressing a native metabolite by the recombinant cell, wherein the native metabolite is selected from the group consisting of chorismate, para-amino-phenylpyruvate (pA-Pyr) and para-nitro-phenylpyruvate (pN-Pyr);
(b) expressing the one or more heterologous enzymes; and
(c) converting the native metabolite to the pN-Phe in the recombinant cell.

13. The method of claim 12, wherein the native metabolite is the chorismate, wherein the one or more heterologous enzymes comprise PapA, PapB and PapC, further comprising
(a) expressing the PapA, the PapB and the PapC by the recombinant cell;
(b) converting the chorismate to para-amino-phenylpyruvate (pA-Pyr) in the recombinant cell;
(c) expressing an N-monooxygenase by the recombinant cell; and
(d) converting the pA-Pyr to para-nitro-phenylpyruvate (pN-Pyr) in the recombinant cell.

14. The method of claim 12, wherein the native metabolite is the chorismate, wherein the one or more heterologous enzymes comprise PapA, PapB and PapC, further comprising
(a) expressing the PapA, the PapB and the PapC by the recombinant cell;
(b) converting the chorismate to para-amino-phenylpyruvate (pA-Pyr) in the recombinant cell;
(c) expressing an aminotransferase by the recombinant cell;
(d) converting the pA-Pyr to para-amino-L-phenylalanine (pA-Phe) in the recombinant cell;
(e) expressing an N-monooxygenase by the recombinant cell; and
(f) converting the pA-Phe to the pN-Phe in the recombinant cell.

15. A method of producing a target polypeptide having para-nitro-L-phenylalanine (pN-Phe) in the recombinant cell of claim 1, wherein the recombinant cell comprises the target polypeptide, comprising
(a) expressing a heterologous amino-acyl tRNA synthetase and a transfer RNA in the recombinant cell; and
(b) incorporating the pN-Phe into the target polypeptide in the recombinant cell without requiring exposure of the recombinant cell to exogenous pN-Phe, whereby the target polypeptide having the pN-Phe is produced.

16. The method of claim 15, wherein the target polypeptide having the pN-Phe is secreted by the recombinant cell.

17. The method of claim 15, wherein the target polypeptide having the pN-Phe is on the surface of the recombinant cell.

18. The method of claim 15, wherein the target polypeptide having the pN-Phe is at least 50% more immunogenic than the target polypeptide without the pN-Phe.

19. The method of claim 15, further comprising growing the recombinant cell in a culture medium having glucose as the sole carbon source for the recombinant cell, wherein the culture medium is not supplemented with exogenous pN-Phe.
